(19) United States
(12) Patent Application Publication
(10) Pub. No.: US 2023/0357850 A1
(43) Pub. Date: Nov. 9, 2023

(54) TREATMENT OF AUTONOMIC DYSFUNCTION RESULTING FROM OPIOID USE

(71) Applicant: SMITH GENETICS RESEARCH LLC, Upperville, VA (US)

(72) Inventor: Douglas R. Smith, Upperville, VA (US)

(21) Appl. No.: 18/218,645

(22) Filed: Jul. 6, 2023

Related U.S. Application Data
(63) Continuation-in-part of application No. 17/722,281, filed on Apr. 15, 2022.
(60) Provisional application No. 63/175,733, filed on Apr. 16, 2021.

(51) Int. Cl. C12Q 1/6883

(52) U.S. Cl. CPC C12Q 1/6883; C12Q 2600/106; C12Q 2600/154

(57) ABSTRACT

A method of treatment of a subject suffering from autonomic dysfunction due to opioid use is described. The autonomic dysfunction can be determined by, for example, detecting genetic damage, such as DNA hypermethylation, measuring catecholamine concentrations, collecting vitals data, and/or surveys. The method of treatment can include administering a partial opioid agonist, such as buprenorphine.

### CROSS REFERENCE TO RELATED APPLICATIONS

This application is a Continuation-in-Part (CIP) of U.S. application Ser. No. 17/722,281 filed on Apr. 15, 2022 which claims the benefit of U.S. Provisional Application No. 63/175,733 filed on Apr. 16, 2021, both of which are incorporated by reference as if fully set forth herein.

### TECHNICAL FIELD

Described herein is a method of treatment of individuals suffering from autonomic dysfunction, more specifically, a method for treatment of individuals suffering from autonomic dysfunction due to genetic damage resulting from opioid use by administering a partial opioid agonist. In certain embodiments, buprenorphine is used in the treatment of autonomic dysfunction caused by opioid use and subsequent cessation and/or reduction of use.

### BACKGROUND

The class of medications known as opioids are either prescribed medications most often used to control pain, or purchased illegally, such as heroin. These drugs can be abused, whether they are legally obtained with a prescription from the doctor, or illegally obtained on the streets.

Opioids have been in use by humans for generations. This class of medications is defined by the ability of a compound to bind to any of the known opioid receptors in the body and produce either an agonist or partial agonist effect. For years, a recognized risk of opioid use was believed to be a condition called Opioid Use Disorder (“OUD”), otherwise referred to as Opioid Addiction or Opioid Dependency. Opioid addiction (as it has been termed) was labeled a brain disease under the Brain Disease Model of Addiction and hypothesized as early as 1988 to involve the neurotransmitter dopamine. The theory is that addiction is a disorder of the dopamine neurotransmitter system. In essence, addictions increase dopamine to such an extent that once the drug or the stimulus is gone, the body is unable to replicate the same amount of dopamine naturally.

The National Institutes of Health projects that nearly 50 million adults in the United States alone have chronic or severe pain with over 25 million American adults reporting chronic daily pain in the past 3 months. While the overall national opioid dispensing rate declined between 2012 to 2019, in 2019, the dispensing rate had fallen to the lowest in the 14 years. In 2019, 46.7 prescriptions were issued per 100 persons, which totals more than 153 million opioid prescriptions being issued in 2019. Currently the U.S. market for opioids for chronic pain management is estimated to be on the order of $10 billion.

One of the organizations involved in the field is the American Society of Addiction Medicine (“ASAM”). ASAM's definition of addiction, which was adopted in September of 2019, is “addiction is a treatable, chronic medical disease involving complex interactions among brain circuits, genetics, the environment, and an individual's life experiences.” ASAM further provides that “people with addiction use substances or engage in behaviors that become compulsive and often continue despite harmful consequences.”

The four foundations of the above definition are brain circuits, genetics, the environment, and life experiences. Notably, the ASAM definition is merely an adopted definition backed by little scientific evidence. The scant evidence is clear from an article published on ASAM's website. Nauts, M. D., D. “The ASAM Treatment of Opioid Use Disorder Course—Disclosure Information,” FASAM (Sep. 11, 2019).

The Diagnostic and Statistical Manual of Mental Disorders, Fifth Edition (“DSM-5”) is the standard classification of mental disorders used by mental health professionals in the United States. Opioid use disorder (“OUD”) is a disease characterized by a problematic pattern of continued opioid misuse. According to the DSM-5, opioid use disorder involves the repeated occurrence of two or more of eleven identified problems within a twelve-month period that include (1) opioid withdrawal symptoms; (2) failing job, school or home responsibilities due to recurrent opioid use; (3) unsuccessful efforts to control opioid use; (4) opioid use in longer or in larger amounts than anticipated; (5) excessive amount of time spent getting or using the opioid, or recovering from its effects; (6) opioid tolerance; (7) opioid use in physically dangerous situations; (8) craving or strong desire to use opioids; (9) social or interpersonal problems caused by opioid effects; (10) giving up on occupational, recreational or social activities due to opioid use; and (11) continued opioid use despite knowledge of the addiction problem. Sever opiate dependance exists when six or more of these problems exist.

Anxiety and depression are common among patients having chronic pain and are risk factors for prescription opioid abuse and overdose. Indeed, people suffering from chronic pain are four times as likely to have anxiety or depression than those without chronic pain. Gureje et al., Persistent pain and well-being: a World Health Organization Study in Primary Care, JAMA, 1998; 280(2): 147:51.

Opioid addiction exists when someone becomes dependent on opiates beyond the need to control the pain and feels a compulsive need to continue using the drugs despite numerous attempts to quit, and despite knowing that opiate use will have negative consequences. Opioid dependence is not the same as addiction. Indeed, patients suffering from OUD benefit from Medication Assisted Therapy (MAT) for long-term maintenance to prevent relapse after a medically supervised cessation of use (detoxification). MAT is a multi-pronged approach that combines approved medications with counseling and support to treat patients suffering from OUD. Methadone, buprenorphine, buprenorphine-naloxone, and naltrexone are all approved for this use.

In particular, buprenorphine and buprenorphine in combination with naloxone are currently FDA-approved in a variety of formulations for sublingual administration for the treatment of OUD, opioid “withdrawal”, and chronic pain. Current FDA-approved buprenorphine formulations for opioid use disorder contain buprenorphine in combination with the inactive ingredient naloxone which is included with the aim of deterring abuse of buprenorphine via the intravenous route. Buprenorphine is a partial opioid agonist that has a lower risk of overdose compared to full opioid agonists (e.g., morphine, hydrocodone, methadone, oxycodone) due to what is referred to as a “ceiling effect” on respiratory depression due to buprenorphine's activity as a partial opioid agonist.

Notwithstanding, at the current time, the overwhelming majority of these patients are being misdiagnosed resulting in improper treatment, for example, therapy or counseling at the expense prescribing a partial opioid agonists, such as buprenorphine. The error occurs because providers frequently diagnose the opioid user with mental health condition prior to examining whether there is a physical health condition requiring a different treatment approach.

Rather, “opioid craving” more accurately describes the condition than opioid use disorder or opioid addition. Opioid craving (“OC”) is both a symptom and a driver of the pathological consumption of opioids positioning OC a critical treatment target. According to the questionable Brain Disease Model of Addiction, OC is a consequence of a surge in dopamine in response to opioid use inducing neuroadaptations, impeding self-control, and promoting drug-seeking behavior. However, replicating studies have demonstrated no surge in brain dopamine in response to opioid. Thus, a serious question exists as to whether OC is merely a consequence of abnormal brain processes or are there other physiological, anatomical, and/or molecular causes.

The inventors propose that OC is a sequelae of an unendurable autonomic dysfunction resulting from opioid abstinence. Additionally, catecholamine toxicity (e.g., norepinephrine and epinephrine) is believed to be directly associated with autonomic dysfunction. Moreover, elevated methylation within the human OPRM1 promoter region of opioid users is believed to be the molecular level cause autonomic dysfunction and associated symptoms.

There remains a need in the art for the accurate diagnoses and treatment of those individuals suffering from autonomic dysfunction caused by opioid use and subsequent cessation/reduction.

### SUMMARY

The present invention relates to methods, materials, and kits for the treatment of autonomic dysfunction by, for example, administering an opioid agonist, partial opioid agonist, or combination formulation. Treatment with full agonist and partial opioid agonists are possible. The full opioid agonists offer partial and temporary relief from symptoms but with risks. The partial agonist, buprenorphine, maintains a more complete and longer lasting relief with a higher level of safety due to the ceiling effect previously described.

Other treatments are also contemplated, such as off-site monitoring. The current methods and compositions provide methods and kits related to diagnosing and treating subjects (e.g., patients with a history of opioid use/abuse, an opioid addiction, habit or dependency) who suffers from autonomic dysfunction. In certain embodiments of the invention, the subject suffers from autonomic dysfunction caused by opioid use and subsequent cessation of said use. In other embodiments, the subject suffers from autonomic dysfunction caused by opioid use and subsequent substantial reduction of said use.

An aspect of the invention provides a method of treatment for a subject suffering from autonomic dysfunction comprising initially evaluating the subject and administering or prescribing buprenorphine to treat the subject on a needed basis. In certain embodiments of the invention, the subject is treated with buprenorphine daily (daily basis), every two days (2-day basis), every week (weekly basis), every two weeks (2-week basis), every month (monthly basis) or any other time period determined to be necessary in the treatment of the subject.

In one embodiment, the autonomic dysfunction is a subtype or type of a neuroendocrine emergency. Still further to this embodiment of the invention, the neuroendocrine emergency is an elevated state of activation or hyperstimulation of the sympathetic nervous system and the parasympathetic nervous system.

In another embodiment, the method of treatment for the subject suffering from autonomic dysfunction may additionally include monitoring the subject. In certain embodiments of the invention, the subject may be monitored on a periodic basis. Further pursuant to this embodiment of the invention, the periodic basis is at least on a weekly basis.

In other embodiments, the subject is monitored on a real-time basis. Further pursuant to this embodiment of the invention, monitoring the subject is through a direct detection of laboratory values in a plurality of bodily tissues and fluids. Still further pursuant to this embodiment, monitoring the subject may instead or additionally be through an indirect detection of any one or more of a plurality of bodily measurements.

In another embodiment, the bodily measurement may be a heart rate variability. Further pursuant to this embodiment of the invention, the heart rate variability is measured through photoplethysmography.

In still other embodiments, recommendations for treatment of the subject and determination of whether said subject suffers from autonomic dysfunction can be based upon any one or more of a plurality of bodily measurements including catecholamine concentrations or levels, such as epinephrine and/or norepinephrine. In one embodiment, an elevated catecholamine level in the subject is indicative of autonomic dysfunction. Catecholamine levels in subjects can be compared to a reference value, reference range, or control in order to determine whether the patient suffers from autonomic dysfunction and before treatment administration.

In another embodiment, an assay is performed to determine whether the subject exhibits elevated DNA methylation. More particularly, the methylation assay can measure the degree of methylation within the human OPRM1 gene. Even more particularly, the methylation assay can measure the degree of methylation within the promoter region of the human OPRM1 gene. In another embodiment, the methylation assay can measure the degree of methylation within the CpG Island of the human OPRM1 promoter region. In yet another embodiment, the methylation assay can measure the degree of methylation at one or more specific CpG sites within the human OPRM1 promoter region. In some embodiments, a subjects methylation level is compared to a reference value, reference range, and/or control (e.g., sample taken from individual with no history of opioid use). Elevated methylation in a subject can be indicative of autonomic dysfunction and determinative of treatment. Elevated methylation may also be indicative defective mu (μ) opioid receptors.

In other embodiments, additional techniques such as administering surveys to test subjects, including but not limited to the Autonomic Dysfunction (or Distress) Scale (ADS) and an Opioid Craving Scale (OCS) may be performed to determine if a subject suffers from autonomic dysfunction. In some embodiments, one or more surveys are performed individually or in conjunction with catecholamine level determination and/or DNA methylation determination. Scores are recorded and may be compared to a reference value, reference range, or control.

In certain embodiments, the method of treatment results in a reduction in autonomic dysfunction. In one embodiment, a subject is treated a full opioid agonist, partial opioid agonist, or combination thereof. In another embodiment, a subject is treated by administering to and/or prescribing the subject with an effective amount of buprenorphine. An effective amount of buprenorphine can be predetermined by a qualified health care provider in situ and can be in a variety of forms including, for example, sublingual tablets, subcutaneous/intramuscular/intravenous injection, or subdermal implant. For example, a therapeutically effective amount (sublingual tablets) for an average adult can be between 1 and 24 mg/day. Alternatively, depending on the subject's physical condition and age, a therapeutically effective amount can be slightly above 24 mg/day and slightly below 1 mg/day.

Another aspect provides a method of treatment of a subject suffering from autonomic dysfunction including the steps of using buprenorphine to treat the subject and monitoring the subject remotely and in real-time. Further pursuant to this embodiment of the invention, the subject is treated using buprenorphine on any one of a daily basis, a two-day basis, a weekly basis, a bi-weekly basis, a monthly basis, and any other time period determined to be necessary in the treatment of the subject. Still further pursuant to this embodiment, the subject being monitored remotely is not in the direct presence of a medical professional and a system that provides an analysis of the monitoring results.

In yet another aspect, provided herein is a method of treatment of a subject suffering from autonomic dysfunction having the steps of monitoring the subject and adjusting an amount of buprenorphine administered or prescribed to treat the subject based upon the data received from the monitoring step. In an embodiment, the monitoring the subject is on a periodic basis. Further pursuant to this embodiment, the periodic basis is at least on a weekly basis.

In another embodiment, the monitoring the subject is on a real-time basis. Further pursuant to this embodiment, the monitoring of the subject is remotely.

Other aspects and embodiments will become apparent upon review of the following description taken in conjunction with the accompanying drawings. The invention, though, is pointed out with particularity by the included claims.

### BRIEF DESCRIPTION OF THE DRAWINGS

Reference will now be made to the accompanying drawings, which are not necessarily drawn to scale, and wherein:

FIG. 1 is an illustration showing normal mu (μ) opioid receptor maintain balance and homeostasis in the autonomic nervous system.

FIG. 2 is an illustration showing normal mu (μ) opioid receptor with all Sp1 binding cites available for binding.

FIG. 3 is an illustration showing binding of Sp1 (Special Protein 1) to the available binding site of the normal mu (μ) opioid receptor.

FIG. 4 is an illustration showing transcriptional machinery becomes attracted to the Sp1 bound to the Sp1 binding site of the normal mu (μ) opioid receptor.

FIG. 5 is an illustration showing all twelve pieces of a protein assimilating to the normal mu (μ) opioid receptor (OPRM1).

FIG. 6 is an illustration demonstrating a normal mu (μ) opioid receptor maintaining balance and homeostasis in the autonomic nervous system.

FIG. 7 is an illustration showing one of the CpG islands of the OPRM1 gene becoming methylated.

FIG. 8 is an illustration showing abnormal mu (μ) opioid receptors with all Sp1 binding sits available for binding except the one blocked by the methyl group.

FIG. 9 is an illustration showing special protein one binds to all Sp1 sites available for binding except the one blocked by the methyl group of the abnormal mu (μ) opioid receptor [0047]

FIG. 10 is an illustration showing transcriptional machinery becomes attracted to the Sp1 bound to the Sp1 binding sites except the one blocked by the methyl group of the abnormal mu (μ) opioid receptor.

FIG. 11 is an illustration showing only eleven of the twelve pieces of a protein assimilating to the abnormal mu (μ) opioid receptor.

FIG. 12 is an illustration demonstrating the abnormal mu (μ) opioid receptor is unable to maintain balance and homeostasis within the autonomic nervous system.

FIG. 13 is an illustration showing the addition of buprenorphine to the abnormal mu (μ) opioid receptor.

FIG. 14 is an illustration showing the abnormal mu (μ) opioid receptor combined with buprenorphine results in a receptor now able to maintain balance and homeostasis within the autonomic nervous system.

FIG. 15 illustrates a map of the human OPRM1 promoter and non-promoter regions with targeted CpG sites for methylation analysis.

FIG. 16A-C shows a table with data from all CpG sites assayed test subjects versus controls.

FIG. 17 shows a table with data from hypermethylated CpG sites.

FIG. 18A-C shows a table with DNA methylation data for each specific test sample and CpG target site.

FIG. 19A-B shows a table with averages and standard deviations from data in FIG. 24.

FIG. 20 shows a table with data for CpG sites within the human OPRM1 promoter.

FIG. 21A-C shows a table with data for all 15 samples and CpG sites within the human OPRM1 promoter.

FIG. 22A-B shows a table with averages and standard deviations from data in FIG. 21.

FIG. 23 is a bar graph illustrating data presented in FIG. 21.

FIG. 24A-C shows a table with data for the 8 test subjects (participants) and CpG sites within the human OPRM1 promoter.

FIG. 25A-B shows a table with averages and standard deviations from data in FIG. 24.

FIG. 26 is a bar graph illustrating data presented in FIG. 24.

FIG. 27A-C shows a table with data for the 6 clinical test subjects (participants) and CpG sites within the human OPRM1 promoter.

FIG. 28A-B shows a table with averages and standard deviations from data in FIG. 33.

FIG. 29 is a bar graph illustrating data presented in FIG. 33.

FIG. 30A-B are correlation plots of methylation percent among specific CpG sites.

FIG. 31A-B is a table with catecholamine assay data and Autonomic Dysfunction Scale/Opioid Craving Scale scores collected prior to buprenorphine administration.

FIG. 32A-B is a table with catecholamine assay data and Autonomic Dysfunction Scale/Opioid Craving Scale scores collected after buprenorphine administration.

FIG. 33 is a table showing mean methylation percentages and standard deviations for the sixteen CpG sites analyzed.

FIG. 34 is a biplot graph prepared from the Principal Component Analysis.

FIG. 35 is a biplot graph prepared from the Non-Metric Multidimensional Scaling Analysis.

FIG. 36 is a table with data from the Mann-Whitney α-Test.

FIG. 37 is a table with data from the Kruskal-Wallace Test.

FIG. 38 is a table with data from the one-way ANOVA Test.

FIG. 39 is a table with data from the PERMANOVA model.

### DETAILED DESCRIPTION

Embodiments of the present invention will be described more fully herein with reference to the accompanying drawings. Preferred embodiments of the invention may be described, but this invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments of the invention are not to be interpreted in any way as limiting the invention.

This document provides methods, materials, and kits for the treatment of a subject suffering from autonomic dysfunction caused by opioid use and subsequent cessation of opioid use. For example, this document provides methods, materials, and kits for determining catecholamine levels in a biological sample obtained from a test subject and/or analyzing methylation of the human OPRM1 promoter region to determine whether the promoter region is hypermethylated in a test subject. The methods, materials, and kits described herein may further compare catecholamine levels and/or OPRM1 promoter region methylation to a normal sample, a control sample, a reference value, or reference range. The methods, materials, and kits described herein may employ additional techniques such as administering surveys to test subjects, including but not limited to the Autonomic Dysfunction (or Distress) Scale (ADS) and an Opioid Craving Scale (OCS) in conjunction with catecholamine level determination and/or DNA methylation determination. Scores are recorded and may be compared to reference values.

The methods, materials, and kits provide for the administration of a treatment of the test subject Treatment may be administration of a therapeutic amount of an opioid agonist “Opioid agonist” is intended to mean full agonists, such as morphine, hydrocodone, methadone, oxycodone, as well as partial agonists, such as buprenorphine. Partial agonists, like buprenorphine, are particularly useful. There is a lower risk of overdose from partial agonist use compared to full opioid agonists due to what is referred to as a “ceiling effect” on respiratory depression.

As used in the specification and in the appended claims, the singular forms “a”, “an”, and “the” include plural referents unless the context clearly indicates otherwise. For example, reference to “an opioid” may include a plurality of such opioids.

It will be understood that relative terms may be used herein to describe one element's relationship to another element as, for example, may be illustrated in the Figures. It will be understood that relative terms are intended to encompass different orientations of the elements in addition to the orientation of elements as illustrated in the Figures. It will be understood that such terms can be used to describe the relative positions of the element or elements of the invention and are not intended, unless the context clearly indicates otherwise, to be limiting.

Embodiments of the present invention are described herein with reference to various perspectives, including, for example, perspective views that are representations of idealized embodiments of the present invention. As a person having ordinary skill in the art would appreciate, variations from or modifications to the shapes as illustrated in the Figures or the described perspectives are to be expected in practicing the invention. Such variations and/or modifications can be the result of manufacturing techniques, design considerations, and the like, and such variations are intended to be included herein within the scope of the present invention and as further set forth in the claims that follow. The articles of the present invention and their respective components described or illustrated in the Figures are not intended to reflect a precise description or shape of the component of an article and are not intended to limit the scope of the present invention.

Although specific terms are employed herein, they are used in a generic and a descriptive sense only and not for purposes of limitation. All terms, including technical and scientific terms, as used herein, have the same meaning as commonly understood by one of ordinary skill in the art to which this invention belongs unless a term has been otherwise defined. It will be further understood that terms, such as those defined in commonly used dictionaries, should be interpreted as having a meaning as commonly understood by a person having ordinary skill in the art to which this invention belongs. It will be further understood that terms, such as those defined in commonly used dictionaries, should be interpreted as having a meaning that is consistent with their meaning in the context of the relevant art and the present disclosure. Such commonly used terms will not be interpreted in an idealized or overly formal sense unless the disclosure herein expressly so defines otherwise.

The class of medications known as the opioids have been in use by humans for generations. This class of medications is defined by the ability of a compound to bind to any of the known opioid receptors in the body and produce either an agonist or partial agonist effect. For years, a recognized risk of opioid use was believed to be a condition called Opioid Use Disorder (“OUD”), otherwise referred to as opioid addiction. As explained below, opioid addiction, as with all addictions, was characterized as a brain disease believed to involve the neurotransmitter dopamine. The American Society of Addiction Medicine's (“ASAM”) definition of addiction (adopted September 2019), is “a treatable, chronic medical disease involving complex interactions among brain circuits, genetics, the environment, and an individual's life experiences (emphasis added).” ASAM further provides that “people with addiction use substances or engage in behaviors that become compulsive and often continue despite harmful consequences.” https://www.asam.orgquality-care/definition-of-addiction. Missing, however, from the ASAM definition are physiological and/or genetic alterations (e.g., mutations, increased methylation) caused by opioid use.

Not surprisingly, the ASAM definition of “addiction” has been consistently applied to “opioid addiction.” According to ASAM, the four fundamentals of addiction are brain circuits, genetics, the environment, and life experiences. However, it is noted that the definition of addiction as put forth by ASAM is an adopted definition with little scientific evidence to back it particularly in relation to so-called diagnosis of opioid addiction. “The ASAM Treatment of Opioid Use Disorder Course—Disclosure Information” by Daniel Nauts, MD, FASAM (Sep. 11, 2019).

The Diagnostic and Statistical Manual of Mental Disorders, Fifth Edition (“DSM-5”) also leans strongly toward mental condition diagnoses versus physiological and/or genetic diagnoses. DSM-5 is the standard classification of mental disorders used by mental health professionals in the United States. DSM-5 provides that the problematic pattern of opioid use leading to clinically significant impairment or distress is manifested by at least two of the following criteria occurring within a 12-month period: opioids are often taken in larger amounts or over a longer period than was intended and a persistent desire exists or an unsuccessful effort to cut down or control opioid use (in many cases demonstrated by a great deal of time that is spent in activities necessary to obtain the opioid, use the opioid, or recover from its effects); craving, or a strong desire or urge to use opioids; recurrent opioid use resulting in a failure to fulfill major role obligations at work, school, or home; continued opioid use despite having persistent or recurrent social or interpersonal problems caused or exacerbated by the effects of opioids; important social, occupational, or recreational activities are given up or reduced because of opioid use; recurrent opioid use in situations in which it is physically hazardous; and continued opioid use despite knowledge of having a persistent or recurrent physical or psychological problem that is likely to have been caused or exacerbated by the substance.

“Autonomic Dysfunction”, “Autonomic Conflict”, and “Autonomic Distress” are used herein interchangeably. The condition occurs when the autonomic nervous system (ANS), which controls functions responsible for well-being and maintaining balance, does not regulate properly. Some of the basic functions controlled by the ANS include heart rate, body temperature, breathing rate, digestion, and sensation. ANS includes both the sympathetic (SANS) and parasympathetic (PANS) autonomic nervous system. The primary responsibility of the SANS is to trigger emergency responses e.g., fight-or-flight responses to stress. The PANS conserves energy and restores tissues for ordinary functions. Autonomic Dysfunction may be characterized by stimulation of SANS and/or PANS producing a variety of physiological conditions or symptoms. Terms that may be used herein to describe this physiological phenomenon (and associated physiological conditions) include but are not limited to catecholamine storm, catecholamine surge, catecholamine toxicity, norepinephrine toxicity, epinephrine toxicity, sympathetic nervous system toxicity, sympathetic nervous system dysfunction, parasympathetic nervous system toxicity, parasympathetic nervous system dysfunction, and neuroendocrine emergency.

“Tolerance” is defined by either of the following: a need for markedly increased amounts of opioids to achieve intoxication or a markedly diminished effect with continued use of the same amount of an opioid.

A “promoter” refers to a regulatory sequence that is involved in binding RNA polymerase to initiate transcription of a gene. A promoter may be an inducible promoter or a constitutive promoter. An “inducible promoter” is a promoter that is active under environmental or developmental regulatory conditions.

According to the contested “dopamine theory of addiction,” dopamine levels increase following the use of amphetamine, cocaine, nicotine, and morphine. The theory evolved around 1988 based upon the Italian study conducted by Chiara et al. Chiara et al., Drugs abused by humans preferentially increase synaptic dopamine concentrations in the mesolimbic system of freely moving rats, Proc. Natl. Acad. Sci. USA, 1988; 85: 5274-5278.

The human brain includes the ventral tegmental area is representative of a group of neurons located close to the floor of the midbrain. These neurons in the ventral tegmental area are the basis of the dopaminergic cell bodies of the mesocorticolimbic dopamine system and other dopamine pathways theorized to be implicated in the reward circuitry of the human brain. The nucleus accumbens is a region in the basal forebrain rostral to the preoptic area of the hypothalamus. Evidence appears to have implicated the long-term synaptic neuroadaptations in glutamatergic excitatory activity of the neurons in the nucleus accumbens shell and/or core medium spiny neurons in response to chronic drug and alcohol exposure. The prefrontal cortex is the cerebral cortex that covers the front part of the frontal lobe. The primary activity of the prefrontal cortex is considered to be orchestration of thoughts and actions in accordance with internal goals, which has been implicated in planning complex cognitive behavior, personality expression, decision making, and moderating social behavior. Dopamine receptors in prefrontal cortex controls the critical aspects of this decision-making area of the human brain. According to the dopamine theory of addiction, these sections are representative of the dopamine pathways or dopaminergic projections of the human brain.

Other sections of the human brain that have not been strongly implicated in the dopaminergic system include the temporal lobe, which is primarily engaged in deriving meanings from sensory inputs received by the human brain; the caudate nucleus (head) in combination with the caudate nucleus (tail) play a vital role in how the brain learns and the subsequent storing and processing of past memories; the putamen in conjunction with the caudate nucleus forms the dorsal striatum and is involved in both learning and movement; the corpus callosum is a bundle of nerve fibers that allow the left and right cerebral hemispheres of the human brain to communicate while the medial forebrain bundle is a fibrous neural pathway that passes along the midline of the forebrain to the hypothalamus, the region that extends rostrally from the ventral tegmental area; the substantia nigra is a structure located in the midbrain that contains high levels of neuromelanin in dopaminergic neurons where the latter are synthesized and plays an important role in the regulation of movements; the pons is a portion of the hindbrain that serves as a communications and coordination center between the two hemispheres of the brain; the locus ceruleus also spelled locus coeruleus is a nucleus in the pons of the brainstem that is involved with physiological responses to stress and panic; the fourth ventricle is one of the four connected fluid-filled cavities within the human brain, collectively known as the ventricular system, wherein the fourth ventricle is located behind the brain stem and in front of the cerebellum; and the cerebellum is located in the hindbrain vertebrates that plays an important role in motor control.

Recent important studies have called the dopamine theory of addiction into question. These later studies employ the highly sensitive Positron Emission Technology (“PET”) scan. Two representative studies are discussed below. The first study is from 2008 led by a team headed by Mark Daglish. The Daglish study showed no surge in brain dopamine levels in response to opioids. Daglish et al., Brain dopamine response in human opioid addiction, Brit. J. Psych., 2008; 193(1): 65-73. The second study is from 2014 led by a team headed by Ben Watson, again utilizing the PET methodology, also failed to show any surge in brain dopamine in response to opioids. Watson et al., Investigating expectation and reward in human opioid addiction with [(11)C] raclopride PET, Addict Biol., 2014; 19(6): 1032-40. FIG. 5 provides a graphical representation as to the theory of addiction via a surge in brain dopamine that has been contested.

Recent scientific evidence questions the mental disorder or brain focused diagnoses discussed above. Rather the results herein support a conclusion that opioid products are defective—causing serious and permanent physiological and/or genetic damage. It is believed that exposure to the opioids causes methylation within the CpG island of the human OPRM1 promoter region resulting in gene silencing and the formation of an abnormal mu (μ) opioid receptor that no longer functions to maintain homeostasis within the autonomic nervous system. Hypermethylation within the OPRM1 promoter region is believed to result in autonomic dysfunction. One of the characteristics of this autonomic dysfunction is catecholamine (e.g., epinephrine) toxicity.

OPRM1 (also called MOR-1, MOR1, MOP, LMOR, etc.) is responsible for the protein called the mu (μ) opioid receptor. Opioid receptors are part of the endogenous opioid system, which regulates pain, reward, and addictive behaviors. Opioid receptors are found in the nervous system and embedded in the outer membrane of neurons. Opioid/receptor binding triggers a cascade of chemical signals in the nervous system that reduce excitability of certain neurons producing feelings of pleasure (euphoria) and pain relief. Increased production of certain neurotransmitters (e.g., dopamine) is also caused by opioid/receptor interaction. The mu (μ) opioid receptor is the primary receptor for endogenous opioids called beta-endorphin and enkephalins, which help regulate the body's response to pain, among other functions. The mu (μ) opioid receptor is also the binding site for many exogenous opioids, such as commonly prescribed pain medications such as oxycodone, fentanyl, buprenorphine, methadone, oxymorphone, hydrocodone, codeine, and morphine, as well as illegal opioid drugs such as heroin.

Normal function of the OPRM1 gene in producing a normal mu (μ) opioid receptor, abnormal function of the OPRM1 gene methylated due to exposure to opioids, and abnormal mu (μ) opioid receptor treated with buprenorphine are discussed below.

### Normal Function of the OPRM1 Gene in Producing a Normal Mu (μ) Opioid Receptor

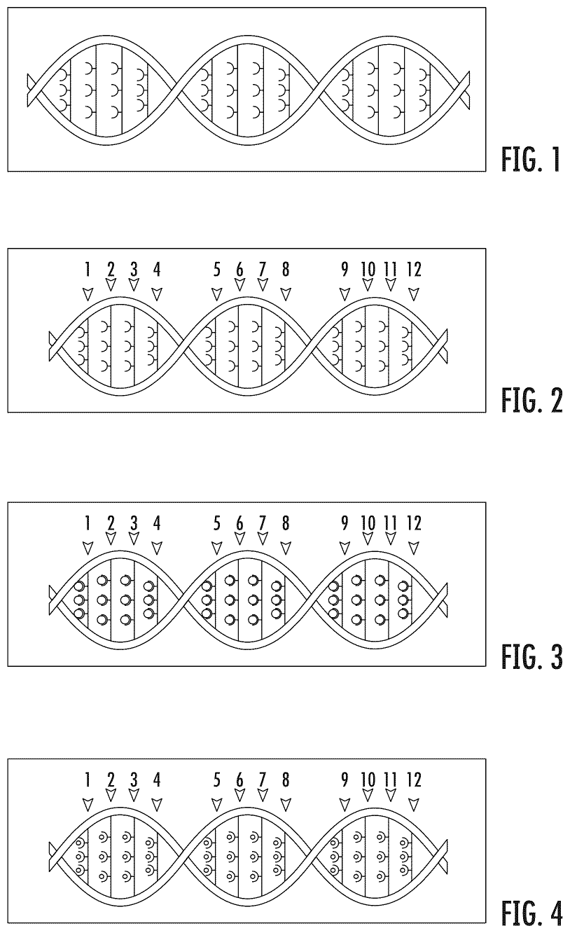

As illustrated in FIG. 1, the normal mu (μ) opioid receptor is able to maintain balance and homeostasis within the autonomic nervous system. FIG. 2 is an illustration showing normal mu (μ) opioid receptor s with all Sp1 binding sits available for binding, while FIG. 3 is an illustration showing special protein one binds to the available binding site of the normal mu (μ) opioid receptor. FIG. 4 is an illustration showing transcriptional machinery becomes attracted to the Sp1 bound to the Sp1 binding site of the normal mu (μ) opioid receptor. FIG. 5 is an illustration showing all twelve pieces of a protein assimilating to the normal mu (μ) opioid receptor following transcription. FIG. 6 is an illustration demonstrating a normal mu (μ) opioid receptor maintaining balance and homeostasis in the autonomic nervous system.

Abnormal Function of the OPRM1 Gene Methylated from Opioid Exposure

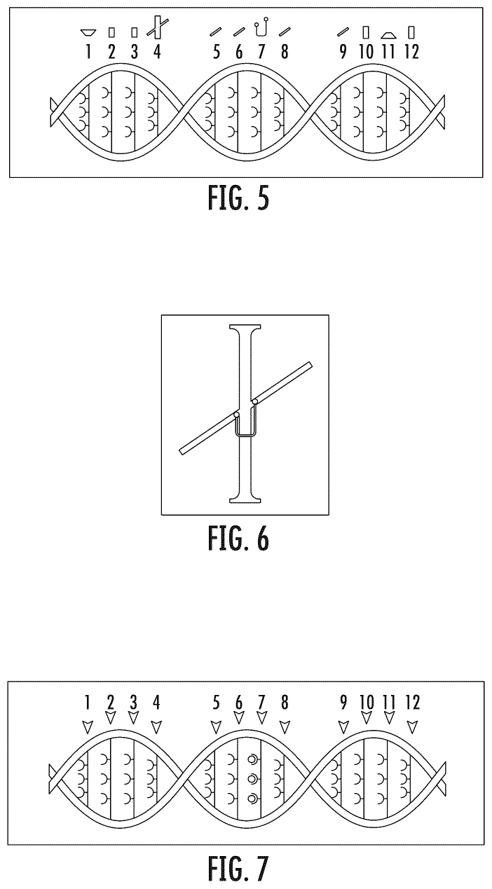

FIG. 7 is an illustration showing one of the CpG islands of the OPRM1 gene becoming methylated. This methylation may produce “partial gene silencing” whereby a receptor is produced, but it is an abnormal receptor with impaired function. As further discussed herein, the abnormal mu (μ) opioid receptor is no longer able to maintain balance and homeostasis within the autonomic nervous system.

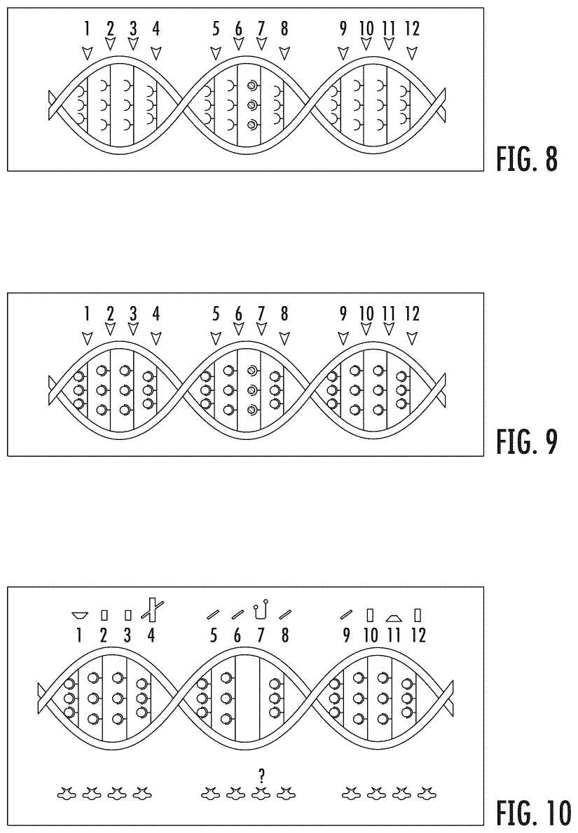

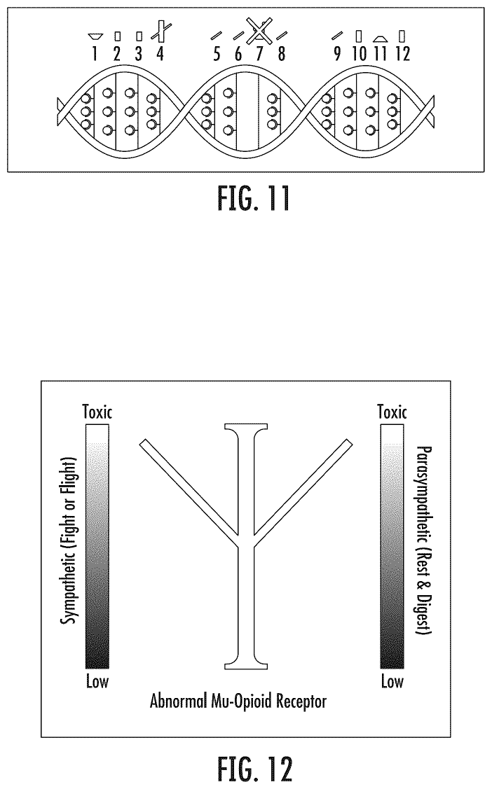

FIG. 8 is an illustration showing abnormal mu (μ) opioid receptor s with all Sp1 binding sits available for binding except the one blocked by the methyl group. FIG. 9 is an illustration showing special protein one binds to all Sp1 site available for binding except the one blocked by the methyl group of the abnormal mu (μ) opioid receptor. FIG. 10 is an illustration showing transcriptional machinery becomes attracted to the Sp1 bound to the Sp1 binding sites except the one blocked by the methyl group of the abnormal mu (μ) opioid receptor. Inasmuch, FIG. 11 is an illustration showing only eleven of the twelve pieces of a protein assimilating to the abnormal mu (μ) opioid receptor. Finally, FIG. 12 illustrates how the abnormal mu (μ) opioid receptor is unable to maintain balance and homeostasis within the autonomic nervous system when opioid abstinence is attempted. This dysfunction within the autonomic nervous system results in a true neuroendocrine emergency known as autonomic dysfunction. This autonomic dysfunction is reflected in the abnormal activity in both branches of the autonomic nervous system, the sympathetic nervous system and the parasympathetic nervous system. Autonomic dysfunction, and associated epinephrine toxicity, is a condition of extreme duress and cannot long be endured by the human body.

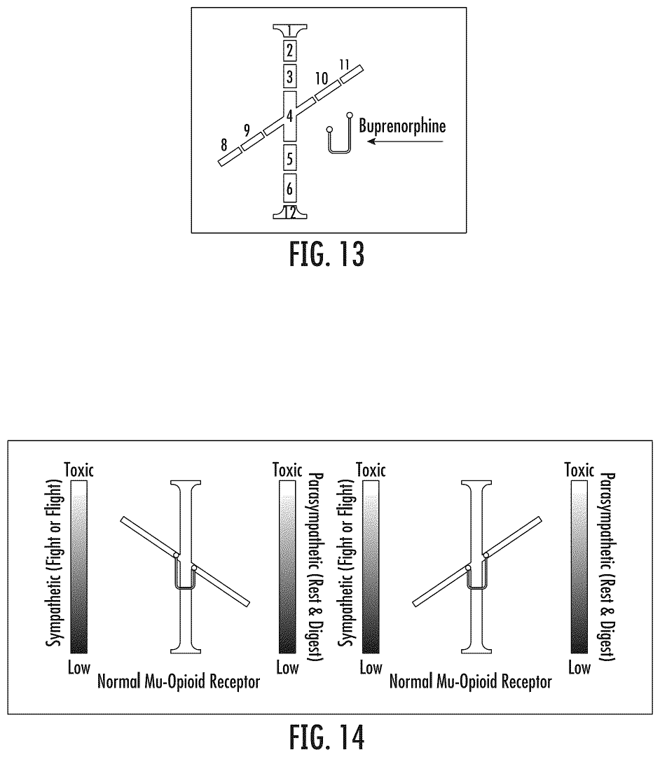

With partial gene silencing, a population of the mu (μ) opioid receptor may remain at or near normal, but these mu-opioid receptors have been rendered as abnormal by the methylation. In other words, the receptor was encoded and generated, but a part of the protein is defective or missing substantially impairing normal function. Partial gene silencing may result in a target molecule to be altered structurally, functionally, and structurally. FIG. 13 illustrates the theoretical effect of buprenorphine on an abnormal mu (p) opioid receptor produced by partial gene silencing resulting in formation of a functional receptor again that can maintain ANS homeostasis. FIG. 14 illustrates the theoretical interaction between an abnormal mu (μ) opioid receptor combined with buprenorphine.

The human body cannot long endure autonomic dysfunction with a catecholamine surge (e.g., epinephrine toxicity) without experiencing a significant degree of suffering. The full opioid agonists offer temporary and partial relief from autonomic dysfunction, but with the common associated risks, such as overdose. Partial opioid agonists, however, such as buprenorphine, offer a longer and more definitive relief from this state of autonomic dysfunction but without the higher risk due to the ceiling effect of buprenorphine. Left untreated, autonomic dysfunction is believed to put the individual at risk of developing life-threatening opioid induced adrenal insufficiency and catastrophic cardiovascular morbidity and mortality.

Existing scientific evidence suggests an association between opioid use and methylation of the CpG Islands within the promoter region of the OPRM1 gene. The scientific evidence further supports a correlation between the methylation of the CpG islands within the promoter region of the OPRM1 gene and the severity of symptoms experienced during the autonomic dysfunction caused by opioid use and cessation/reduction of said use. This evidence at the very least demonstrates that opioid exposure causes methylation of the CpG Islands within the promoter region of the OPRM1 gene. What has not been shown conclusively is the association between autonomic dysfunction, elevated DNA methylation, and catecholamine surge. Below is a summary of that evidence. Assuming the methylation produces gene silencing, one might expect there to be a down regulation of the OPRM1 gene. However, as will be explained below, that does not appear to be the case.

In 2009, direct sequencing of bisulfite-treated DNA showed that the percent methylation at two CpG sites was significantly associated with heroin addiction. Nielsen, D. A., et al., Increased OPRM1 DNA Methylation in Lymphocytes of Methadone-Maintained Former Heroin Addicts, Neuropsychopharmacology, 2009, 34: 867-873. In 2011, researchers found an association between increased methylation in the OPRM1 gene and opioid dependence. Methylated CpG sites located in OPRM1 promoter may block the binding of Sp1 and other transcription activators. Chorbov, V. M. et al., Elevated Levels of DNA Methylation at the OPRM1 Promoter in Blood and Sperm from Male Opioid Addicts, J. Opioid Manag., 2011; 7(4): 258-264.

In 2014, a research team found an association between increased methylation within the OPRM1 promoter neonatal abstinence syndrome (NAS) outcomes. Wachman, E. M. et al., Epigenetic Variation in the Mu-opioid Receptor Gene in Infants with Neonatal Abstinence Syndrome, J. Pediatrics, 2014, 166(3): 472-478. In 2018, the same group replicated the previous findings, showing once again that higher levels of OPRM1 methylation, this time at specific CpG sites, are associated with increased NAS severity. Wachman, E. M. et al., Epigenetic Variation in OPRM1 Gene in Opioid-Exposed Mother-Infant Dyads, Genes, Brain an Behavior, 2018, 17(7): e12476.

There is direct evidence that the exposure to the opioids themselves causes the methylation within the CpG Island of the promoter region of the OPRM1 gene. In 2020, Jose Vladimir Sandoval-Sierra in an article entitled “Effect of Short-Term Prescription Opioids on DNA Methylation of the OPRM1 Promoter” shows that the hypermethylation of the OPRM1 promoter is in response to opioid use and that epigenetic differences in OPRM1 and other sites are associated with a short-term use of therapeutic opioids. Sandoval-Sierra, Jose V. et al., Effect of Short-Term Prescription Opioids on DNA Methylation of the OPRM1 Promoter, Clinical Epigenetics, 2020, 12:76. And it is this study that has provided the scientific evidence for causation i.e., it is the ingestion of the opioid into the human body that is causing methylation.

Scientific evidence exists showing a link between opioid use and methylation occurring within the CpG Island of the OPRM1 promoter region. One would expect from the above referenced literature, that an individual suffering from the methylation in the promoter region of the OPRM1 gene would have fewer numbers of the mu (μ) opioid receptors. Such a reduction in the number of mu (μ) opioid receptors would be known as a down-regulation. It was somewhat surprising then that He et al. (2016) did not find this to be the case. According to He et al., “numerous studies have demonstrated no substantial downregulation in the number of MORs (mu-opioid receptors) even in profoundly tolerant animals (for example, De Vries et al. 1993, Simantov et al. 1984; reviewed in Williams et al. 2001). Hence, it is unlikely that tolerance to morphine is mediated solely by desensitization and downregulation of the receptor.” He et. al., Regulation of Opioid Receptor Trafficking and Morphine Tolerance by Receptor Oligomerization, Cell, 2016, 108(2): 271-282.

As shown herein, opioids are really a defective product causing opioid induced genetic toxicity or genetic damage versus the dopamine theory of addiction is substantial. Likewise, the evidence against the conventional opioid addiction label is overwhelming, yet the mental health diagnosis of opioid addiction/dependency/use disorder is applied almost ubiquitously resulting in the administration of improper treatment regimens (i.e., behavioral treatment or therapy versus administration or prescription of opioid agonist or partial agonist, such as buprenorphine).

The recognition that the underlying disorder is not a mental health diagnosis, but a medical diagnosis is essential. Autonomic dysfunction is a physical state characterized by hyper-stimulation or activation of the autonomic nervous system—sometimes both sympathetic and parasympathetic branches. This may also be referred to as neuroendocrine emergency, a highly uncomfortable state for those afflicted producing significant alterations in behavior. It was this behavior that was misinterpreted as a state of addiction or a type of mental illness. Autonomic dysfunction is generally associated with genetic defects, for example, the autonomic dysfunction associated with Familiar Dysautonomia. The inventors propose that autonomic dysfunction associated with opioid use is induced (at least in part) by methylation changes within the OPRM1 promoter region (or other genetic damage) caused by opioid exposure. The invention includes the administration of an opioid agonist or partial agonist (e.g., buprenorphine) for the treatment of autonomic dysfunction caused by opioid use and subsequent abstinence, cessation, or reduction. The inventive indication for the use of buprenorphine is for the medical diagnosis autonomic dysfunction caused by opioid use and subsequent abstinence, cessation, or reduction. Another medical diagnosis may be adult abstinence syndrome with autonomic dysfunction.

As stated above, autonomic dysfunction is a state of a neuroendocrine emergency. The neuroendocrine emergency is an abnormally elevated state of activation of one or both branches of the autonomic nervous system—the sympathetic nervous system and the parasympathetic nervous system. The neuroendocrine emergency known as autonomic dysfunction is a highly uncomfortable state of being. Individuals will go to lengths to stop the symptoms. It was this behavior that was misinterpreted as a state of addiction or a type of mental illness. Buprenorphine provides a relief from this neuroendocrine emergency and the autonomic dysfunction. The endpoint for the use of the buprenorphine will be a reduction in the level of autonomic dysfunction.

Typically, the test subject (e.g., patient) may be administered or prescribed an opioid agonist or partial agonist, such as buprenorphine on a daily basis, but any amount of time determined to be necessary by a qualified health care provider may be used including, but not limited to, a bi-daily basis, a daily basis, every two days, a bi-weekly basis, a weekly basis, a bi-monthly basis, a monthly basis. Moreover, the treatment duration may be any length of time determined to be necessary by qualified medical professionals for the treatment of the subject. In addition to using buprenorphine on some predetermined basis, the subject will undergo and initial evaluation. Furthermore, monitoring of the subject will occur on an ongoing basis. This monitoring will be specifically driven towards the end point of a reduction in the level of autonomic dysfunction. In certain embodiments of the invention, the monitoring of the subject can occur periodically on at least a daily basis, at least a weekly basis, at least a biweekly basis, at least a monthly basis, and any other periodic basis that is appropriate for the treatment of the subject. In other embodiments of the invention, the monitoring may be on a real-time basis. In still other embodiments of the invention, monitoring the subject may be remotely. As used herein, remotely is intended to mean not in the direct presence of the medical personal and/or the system that provides analysis of the results.

According to the present invention, autonomic dysfunction caused by opioid use and cessation can be detected, measured, and monitored in a variety of ways. In certain embodiments of the invention, the monitoring can include direct detection and monitoring of laboratory values in biological samples (e.g., tissues and/or fluids), for example, catecholamine (e.g., epinephrine or norepinephrine) concentrations or levels. Biological samples taken from a test subject, includes, but is not limited to a blood sample, saliva sample, urine sample, tissue biopsy or section, lavage, swab, scrape, aspirate, or other composition that may be extracted from the body. In particular embodiments, the present invention concerns blood or saliva samples.

Biological samples from test subjects can be assayed for catecholamine levels or concentrations. “Catecholamine” is intended to mean any group of amines derived from catechol that have important physiological effects as neurotransmitters and hormones, including but not limited to epinephrine, norepinephrine, and dopamine. In some embodiments, the catecholamines tested for the purpose of determining whether a test subject has autonomic dysfunction are selected from epinephrine, norepinephrine, and/or dopamine. Assays for determining catecholamine levels in biological samples are well known in the art. Exemplary protocols include those described in Chernecky et al., Laboratory Tests and Diagnostic Procedures, 6^{th}, ed., St. Louis, MO: Elsevier Saunders; 2013:302-305; J. Diamant et al., A Precise Catecholamine Assay for Small Plasma Samples, J. Lab. Clin. Med., 85(4):678-693.

Normal ranges for catecholamine levels in adults (18 years and older) include 0 to 62 pg/mL (epinephrine), 0 to 874 pg/mL (norepinephrine), and 0 to 48 pg/mL (dopamine). In an embodiment, the normal ranges can be used as reference values. In some embodiments, a catecholamine level in a test subject (patient who has ceased using opioids) above a reference value or above the normal range (“elevated catecholamine level”) is indicative of autonomic dysfunction caused by opioid use and subsequent cessation. In one embodiment, two or more catecholamines showing elevated levels is indicative of autonomic dysfunction. In yet another embodiment, elevated levels of epinephrine and norepinephrine (combined) is indicative of autonomic dysfunction.

In another embodiment, biological samples from test subjects can be assayed to determine if a test subject has elevated methylation (“hypermethylation”) within the human OPRM1 promoter region. Methylation can be assayed across the entire human OPRM promoter region or across a particular portion of the promoter region, such as the CpG island. Site specific methylation may also be assayed, for example, the methylation assay may concentrate on one or more specific CpG sites within the promoter region to determine whether elevated methylation exists at the site(s). “CpG sites” is intended to mean regions of DNA where a cytosine nucleotide is followed by a guanine nucleotide in the linear sequence of bases along its 5′ to 3′ direction. CpG sites occur with high frequency in genomic regions called CpG islands. Cytosines in the CpG dinucleotide can be methylated to form 5-methylcytosine. In some embodiments, between 1 and 30 CpG sites are assayed.

Examples of methylation assays can fall into the following categories: absolute DNA methylation assays that provide a quantitative measure of DNA methylation levels at single-CpG resolution, relative DNA methylation assays that measure DNA methylation by comparing samples to a suitable reference, global DNA methylation assays that measure a sample's total DNA methylation content, and immunoquantification of global DNA methylation (Immunoquant) that use a modified enzyme-linked immunosorbent assay (ELISA) with an antibody against 5-methylcytosine to quantify the total amount of methylated DNA in a given sample.

Examples of absolute DNA methylation assays include but are not limited to: (i) amplicon bisulfite sequencing (AmpliconBS) uses next-generation sequencing (NGS) of pooled PCR amplicons derived from bisulfite-converted DNA; (ii) Enrichment bisulfite sequencing (EnrichmentBS) is similar to AmpliconBS in its use of bisulfite conversion and NGS, but it uses highly scalable techniques such as padlock probes or microdroplet-based amplification to enrich many genomic regions in parallel rather than relying on separate PCRs for each individual region; (iii) Mass spectrometric analysis of DNA methylation (EpiTyper) combines bisulfite conversion, in vitro transcription and uracil-specific cleavage with mass-spectrometry-based quantification of fragment lengths; and (iv) Bisulfite pyrosequencing (Pyroseq) applies sequencing by synthesisto single PCR amplicons obtained from bisulfite-converted DNA.

Examples of relative DNA methylation assays include but are not limited to: (i) MethyLight uses PCR amplification of bisulfite-converted DNA in combination with fluorescently labeled probes that hybridize specifically to a predefined DNA methylation pattern, typically that of fully methylated DNA; (ii) Methylation-specific melting assays, including methylation-sensitive high-resolution melting (MS-HRM) and methylation-specific melting curve analysis (MS-MCA), apply melting curve analysis to amplicons obtained from bisulfite-converted DNA, which provides a semiquantitative measure of cytosines that have been converted to thymines; and (iii) Quantitative methylation-specific PCR (qMSP) uses DNA-methylation-specific primers in combination with real-time PCR to compare the prevalence of a specific DNA methylation pattern with that of a suitable reference.

Examples of global DNA methylation assays include but are not limited to: (i) High-performance liquid chromatography followed by mass spectrometry (HPLC-MS) quantifies the amount of 5-methylcytosine based on its mass difference compared to unmethylated cytosine; (ii) Immunoquantification of global DNA methylation (Immunoquant) uses a modified enzyme-linked immunosorbent assay (ELISA) with an antibody against 5-methylcytosine to quantify the total amount of methylated DNA in a given DNA sample; (iii) Bisulfite pyrosequencing of repetitive DNA elements (Pyroseq AluYb8/D4Z4/LINE/NBL2) applies pyrosequencing to amplicons obtained from bisulfite-converted DNA using primers that amplify multiple instances of the selected type of repeat which assumes that averaged local DNA methylation levels across specific repetitive regions correlate with global DNA methylation levels.

Other methylation assay examples include the Infinium 450k assay, assays employing emerging technologies e.g., nanopores, nanowire transistors, quantum dots, single-molecule real-time sequencing and atomic force spectroscopy, and genome-wide assays, such as whole-genome bisulfite sequencing, reduced-representation bisulfite sequencing, and methylated DNA immunoprecipitation sequencing or methyl-CpG binding domain enriched sequencing.

For specific CpG sites, methylation percent increases (test sample percent methylation—control percent methylation=percent increase) of between 1 and 10% are considered indicative of autonomic dysfunction. In some embodiments, percent increases of between 2 and 6% and between 3 and 5% are considered indicative of autonomic dysfunction. Likewise an average percent increase of between 1% and 5% calculated from a plurality of CpG sites is considered indicative of autonomic dysfunction. In one embodiment, an average percent increase of between 2.0% and 2.5% is indicative of autonomic dysfunction.

In another embodiment, a methylation percent increase in one or more of the following CpG sites within the OPRM1 promoter region is indicative of autonomic dysfunction: CpG #-22 (cg223700006) (position −336, 5′ of ATG/start codon), CpG #−20*(position −286, 5′ of ATG), CpG #−19*(position −279, 5′ of ATG), CpG #−16*(position −247, 5′ of ATG), CpG #−11 (cg05215925) (position −93, 5′ of ATG), CpG #−8*(position −71, 5′ of ATG), CpG #−7*(position −60, 5′ of ATG), CpG #−5*(position −32, 5′ of ATG), CpG #−4*(position −5, 5′ of ATG), CpG #−2*(position −14, 5′ of ATG), CpG #−1 (cg12838303) (position −10, 5′ of ATG), CpG #8*(position 140, 3′ of ATG), CpG #11*(position 159, 3′ of ATG), CpG #22 (position 328, 3′ of ATG), CpG #29 (position 991, 3′ of ATG), CpG #30 (position 1008, 3′ of ATG), CpG #166 (position 20527, 3′ of ATG), CpG #341 (position 45985, 3′ of ATG), CpG #376 (position 51542, 3′ of ATG), and/or CpG #593 (position 79393, 3′ of ATG). In another embodiment, a methylation percent increase in one or more of the following CpG sites is indicative of autonomic dysfunction: CpG #−1, CpG #−2*, CpG #−4*, CpG #−5*, CpG #−7*, CpG #−8*, CpG #−11, CpG #8, CpG #11, CpG #22, CpG #29, CpG #30, CpG #166, CpG #341, CpG #376, and/or CpG #593. In yet another embodiment, a methylation percent increase in one or more of the following CpG sites is indicative of autonomic dysfunction: CpG #−20*, CpG #−19*, CpG #−16*, CpG #−8*, CpG #−7*, CpG #−5*, CpG #−4*, CpG #−2*, CpG #8, and/or CpG #11. A single methylation assay may target a plurality of predetermined CpG sites, for example, a plurality of those CpG sites within the three groups set forth above (this paragraph). The plurality of target CpG sites for the assay defines an assay panel.

Treatment methods may be applicable to persons suffering genetic damage due to opioid use whether such damage is now known or later discovered. In an embodiment of the invention, the types of genetic damages may be due to the genetic sequence of the nucleotides, known Epigenetic changes, or Epigenetic changes yet to be determined.

In some embodiments, test subjects may be given surveys or questionnaires, such as the Autonomic Dysfunction (or Distress) Scale (ADS) and/or the Opioid Craving Scale (OCS). Both ADS and OCS were specifically designed by the inventors for the purposes of the study described herein. In each case, the queries presented to the test subjects were reviewed by clinicians familiar with the clinical presentations. The Autonomic Dysfunction Scale comprises of a set of 14 conditions that the test subject is asked to score based on their present subjective condition. Responses are graded on a scale of 0 (condition non-existent) to 4 (condition most severe). Measured symptoms are: 1) I am yawning more than normal; 2) My eyes are watering more than normal; 3) My nose is running more than normal; 4) I am having stomach cramping; 5) I am vomiting; 6) I have diarrhea; 7) I am sweating more than normal; 8) The hair on my body is standing on end; 9) My heart is beating hard and fast; 10) I feel anxious; 11) I feel hot then cold; 12) I have a tremor (shaking); 13) I feel like something bad is about to happen; and 14) 1 can't stand feeling this way.

Similarly, the Opioid Craving Scale (OCS) presents the test subject with 7 factors or conditions which the test subject is asked to rank on a scale of 0 (condition non-existent) to 4 (condition most severe). Test subjects score the following: 1) If I had an opioid right now, I would take it; 2) I would not be able to stop myself from taking an opioid right now; 3) I would feel more in control of things if I could take an opioid right now; 4) Taking an opioid right now would make me feel better; 5) If I could take an opioid right now I would feel less restless; 6) I am craving an opioid right now; and 7) Using an opioid right now would make me feel better.

Such surveys, like ADS and OCS may be combined with other assays, such as the OPRM1 promoter methylation assay and/or the catecholamine detection assay described above in order to determine whether a test subject is suffering from autonomic dysfunction.

Other useful processes toward determining whether a subject is suffering from autonomic dysfunction include direct detection and monitoring of the activity of one or both branches (SANS and/or PANS) of the autonomic nervous system. In other embodiments of the invention, the monitoring includes indirect monitoring of the autonomic nervous system by monitoring and taking vital measurements, for example, blood pressure, pulse, and/or respiration. Normal vital sign ranges for average healthy adults while resting are: (1) BP 90/60 mm Hg to 120/80 mm Hg; (2) Respiration/12 to 18 breaths per min; (3) Pulse/60 to 100 beats per min; and (4) Temp/97.8 to 99.1° F. In some embodiments, vital sign measurements can be considered in combination with other assays described herein to determine whether a test subject suffers from autonomic dysfunction. For example, in one embodiments, a test subject is determined to suffer from autonomic dysfunction caused by opioid use and cessation if a test subject has at least one elevated vital sign, at least one elevated catecholamine, OCS>20, and ADS>40. In another embodiment, at least two elevated vital signs, at least two elevated catecholamines, an OCS>20, and an ADS>40 is indicative of autonomic dysfunction caused by opioid use and cessation. In one particular embodiment, the at least two elevated catecholamines are epinephrine and norepinephrine. In another embodiment, OPRM1 promoter methylation may be considered in combination with vital sign data, catecholamine level, and OCS/ADS.

In certain other embodiments of the invention, monitoring is intended to cover all techniques used to monitor autonomic nervous system for the ideal outcome of each subject, for example, heart rate variability. Left untreated, autonomic dysfunction could possibly be a risk factor for the development of opioid induced adrenal insufficiency and catastrophic cardiovascular morbidity and mortality.

In some embodiments, remote monitoring equipment (in home equipment) can be used to monitor a subject's autonomic nervous system. For example, according to certain embodiments, there are a variety of ways to monitor and measure heart rate variability. The invention intends to encompass a multiplicity of methodologies for the measurement of heart rate variability when the results are utilized to assess the status of the autonomic nervous system. According to an embodiment of the invention, the use of a smart phone and a technology known as photoplethysmography (PPG) may be used to determine heart rate variability. In PPG, the workings of the smart phone camera are utilized to detect both transmission through and reflection from the body tissue. Based upon the level of blood perfusion, heart related information can be obtained. From the data collected, a heart rate variability can be calculated. Good heart rate variability is associated with good autonomic nervous system function. The lack of variability in heart rate is associated with abnormal function within the autonomic nervous system. The data may be either uploaded through an App or an Artificial Intelligence (“AI”) device, and logic can then be applied to the data. In an embodiment of the invention, from this process, predetermined recommendations for treatment can be made. Further pursuant to this embodiment of the invention, both ongoing treatment with buprenorphine and a possible tapering off of the amount of buprenorphine to be used can be accomplished. This is vastly superior over any other known current process for monitoring and tapering of a replacement drug therapy.

Monitoring of the autonomic nervous system can used to provide treatment guidance. Any and all technologies and methodologies for monitoring are included in the invention. In certain embodiments, direct measurement of autonomic nerve activity can be used for monitoring. In other embodiments of the invention, laboratory evaluation of tissue or bodily fluids are used for monitoring. In still other embodiments of the invention, indirect methods are used for monitoring. Further pursuant to this embodiment of the invention, heart rate variability may be used in the indirect monitoring of the subject.

According to an aspect of the invention, treatment with buprenorphine and remote real time subject monitoring will dramatically impact the ability to save those with life threatening symptoms. Through advancements in technology, through experience, and with the machine learning capabilities of Artificial Intelligence, utilizing data collected from the subject in real time and during a subject's normal daily activities, enables the subject to be monitored and advised on health issues. According to an embodiment of the invention, these technologies are important in advancing the human life expectancy.

### Example 1: Elevated Catecholamine Levels Indicative of Autonomic Dysfunction

### Example 1a

Catecholamine toxicity (e.g., elevated levels of catecholamine) has the potential for drastic cardiovascular sequela. However, in animal models, not all catecholamines are equal in this regard. For example, epinephrine toxicity was found to result in an increased incidence of cardiac related death. Norepinephrine toxicity had a higher and faster incidence of cardiac related death. The combination, however, was the most lethal in cardiac related deaths producing close to 40% mortality in six hours in mice. Wen-Hsien Lu et al., Toxics 2020, Norepinephrine Leads to More Cardiopulmonary Toxicities than Epinephrine by Catecholamine Overdose in Rats, Toxics, 2020, 8(3):69.

It is proposed that many of the symptoms discussed above are consistent with catecholamine toxicity. Thus, the presence or absence of catecholamine toxicity could be a determining factor in determining whether a subject suffers from autonomic dysfunction during cessation or reduction of use. If the subject suffers from something called opioid addiction (according to the conventional definition), then catecholamine levels during cessation/reduction should be normal. But if opioid addiction is the incorrect diagnosis and, if instead, the subject suffers from autonomic dysfunction, then the catecholamine levels during cessation/reduction should be elevated.

TABLE 1

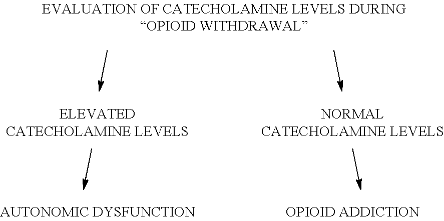

An episode of so-called “opioid withdrawal” may be resolved by the application of a dosage of either a full opioid agonist (i.e., hydrocodone, oxycodone, heroin, or fentanyl) or a partial opioid agonist (i.e., buprenorphine). According to the conventional Brain Disease Model of Addiction and so-called Adhedonia Hypothesis, an agonist relieves adhedonia (e.g., inability to experience pleasure) that is associated, in large part, with a problem in the brain's reward system and dopamine effect, the neurotransmitter that is associated with pleasure.

The inventors contend, however, that the agonist is not resolving a brain function, but rather autonomic dysfunction with catecholamine toxicity. Measurement of catecholamine levels in a test subject before and after the application of a full or partial opioid agonist could determine proper diagnosis. If abnormally elevated catecholamine levels in the test subject are resolved after administration of a full or partial opioid agonist, then opioid addiction with opioid cravings due to “anhedonia” is the incorrect diagnosis. The accurate diagnosis would be Autonomic Dysfunction with opioid craving.

### Case Study

The subject was a Caucasian male aged 37 who had been on prescribed opioids including hydrocodone and oxycodone for an orthopedic condition. Subject had attempted to stop the opioids, but became violently ill and was unable to fully stop. Following standard approval and informed consent protocols, a biological sample (from blood) was obtained both before and two hours after the application of a dosage of buprenorphine.

On arrival in the office, subject was ill-appearing and with initial blood pressure of 138/86, pulse of 88 and was found on examination to have mydriasis, diaphoresis, piloerection, tremor, restlessness, epiphora, excessive yawning, and rhinorrhea. A blood sample was obtained to measure catecholamine levels. Further, subject completed both an Autonomic Dysfunction Scale and the Opioid Craving Scale. Subsequently, the subject was administered 16 milligrams buprenorphine. A second blood sample was obtained after two hours, and both the Autonomic Dysfunction Scale and the Opioid Craving Scale were repeated. With the application of the buprenorphine, all abnormal signs and symptoms resolved including the mydriasis, diaphoresis, piloerection, tremor, restlessness, epiphora, excessive yawning, and rhinorrhea. The catecholamine concentrations and both the Autonomic Dysfunction Scale and the Opioid Craving Scale are as follows:

TABLE 2
Catecholamine Levels Prior to Buprenorphine Administration

| | Current Result and | | Reference |
|---|---|---|---|
| Test | Flag | Units | Interval |
| Catecholamine Frac, P⁰¹ | | | |
| Norepinephrine, PI⁰¹ | 1959/HIGH (Results | pg/mL | 0-874 |
| | confirmed on dilution) | | |
| Epinephrine, PI⁰¹ | 107/HIGH | pg/mL | 0-62 |
| Dopamine, PI⁰¹ | 44 | pg/mL | 0-48 |

As demonstrated in Table 2, the subject experienced what is known as a catecholamine storm with severely elevated norepinephrine and epinephrine levels. Dopamine levels were within the normal range at 44 pg/mL.

TABLE 3
Autonomic Dysfunction Scale Results
Prior to Buprenorphine Administration

| | Scale (0-4) |
|---|---|
| | “0” non-existent and |
| Symptoms | “4” most severe |
| I am yawning more than normal | 4 |
| My eyes are watering more than normal | 4 |
| My nose is running more than normal | 4 |
| I am having stomach cramping | 4 |
| I am vomiting | 0 |
| I have diarrhea | 4 |
| I am sweating more than normal | 4 |
| The hair on my body is standing on end | 4 |
| My heart is beating hard and fast | 3 |
| I feel anxious | 4 |
| I feel hot then cold | 4 |
| I have a tremor (shaking) | 2 |
| I feel like something bad is about to happen | 4 |
| I can't stand feeling this way | 4 |

Prior to buprenorphine administration, the subject experienced extreme autonomic distress reporting the highest severity (4) for 11 out of 14 symptoms. See Table 3.

TABLE 4
Opioid Craving Scale Results Prior
to Buprenorphine Administration

| | Scale (0-4) |
|---|---|
| | “0” non-existent and |
| Craving | “4” most severe |
| If I had an opioid right now, I would | 4 |
| take it | |
| I would not be able to stop myself | 2 |
| from taking an opioid right now | |
| I would feel more in control of things | 4 |
| if I could take an opioid right now | |
| Taking an opioid right now would | 4 |
| make me feel better | |
| If I could take an opioid right now I | 4 |
| would feel less restless | |
| I am craving an opioid right now | 4 |
| Using an opioid right now would | 4 |
| make me feel better | |

Prior to buprenorphine administration, the subject experienced an extreme craving for opioids reporting the highest severity (4) for 6 out of 7 symptoms. See Table 4.

Following buprenorphine administration according to the regimen above, the subject's catecholamine profile reflected dramatically reduced concentrations of norepinephrine (833 pg/mL) and epinephrine (41 pg/mL). Dopamine levels remained within the normal range. Likewise, the subject reported total abatement of autonomic distress and opioid cravings reporting each symptom and craving as “0”.

### Example 1b

A larger study was performed to test the results seen in the single subject case study described above. Following IRB approval and under informed consent, 25 opioid dependent participants stopped taking opioid to induce opioid withdrawal. On the third day, vital signs were obtained at the study site and subjects completed ADS and OCS surveys. Blood samples from the subjects were obtained to assay catecholamine levels. Subsequently, subjects were administered 16 mg buprenorphine sublingually. Two hours after complete absorption of the buprenorphine, the process described above was repeated. Blood samples for catecholamine concentration measurement were also drawn on an equal number of non-opioid dependent individuals as controls.

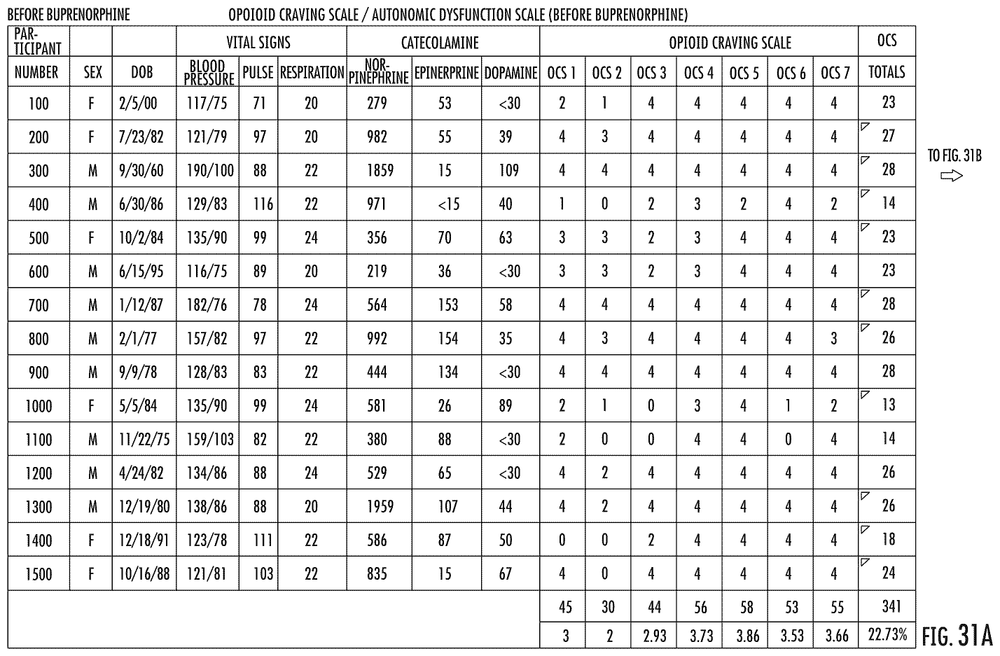

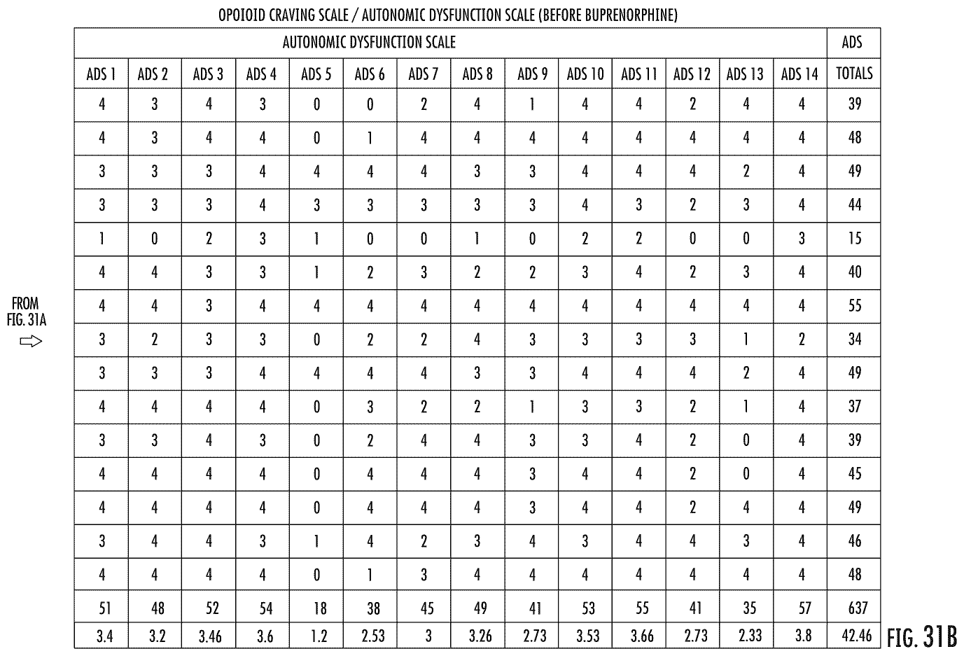

Based on early results, the study was immediately discontinued due to an unacceptable risk of cardiovascular event in the test subjects. While it is recognized that dopamine toxicity and epinephrine toxicity can cause death due to catastrophic cardiovascular events, it was the norepinephrine toxicity, particularly when combined with epinephrine toxicity, that compelled immediate termination of the study. Prior to study discontinuance, the test administrators found that 13 out of 15 subjects had at least one elevated catecholamine. 6 subjects had elevated levels of 2 out of the 3 catecholamines assayed—norepinephrine, epinephrine, and dopamine. 5 participants had elevated norepinephrine levels, 8 had elevated epinephrine levels, and 6 had elevated dopamine levels. 2 participants had extremely elevated levels of norepinephrine (992 pg/mL and 1959 pg/mL) and epinephrine (154 pg/mL and 107 pg/mL), which was particularly troubling to the administrators who discontinued the study primarily on that basis. See FIGS. 31A-B.

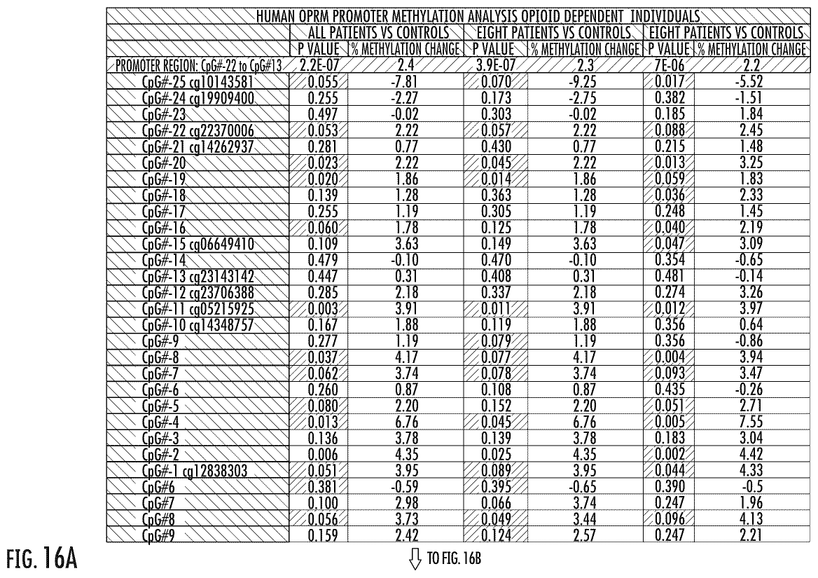

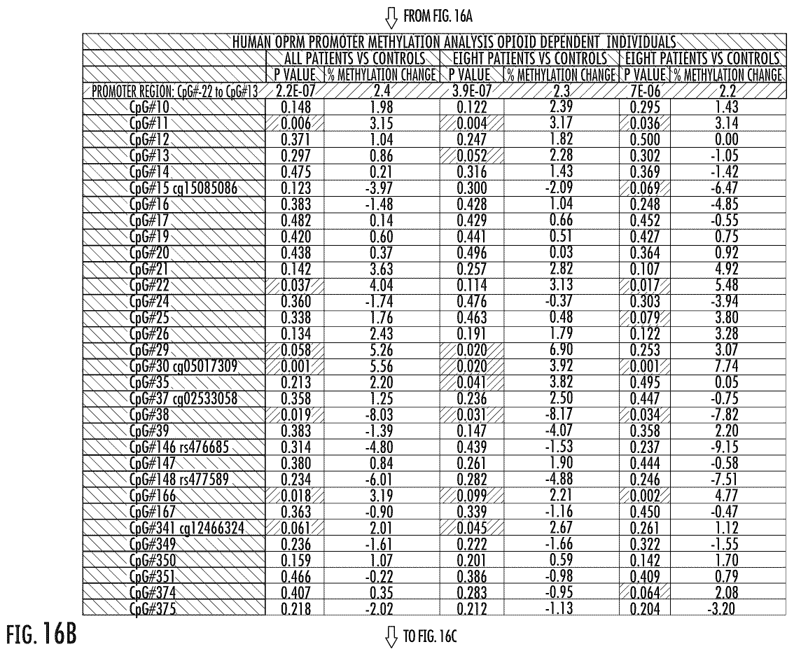

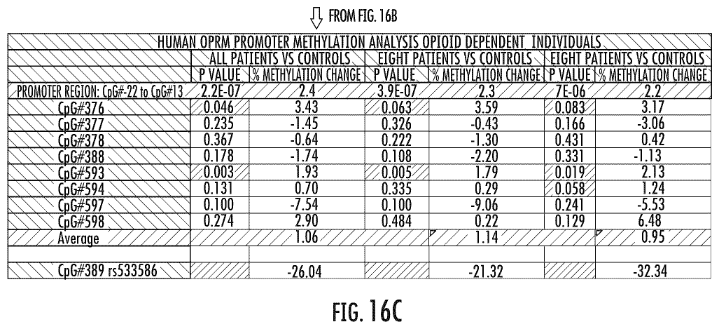

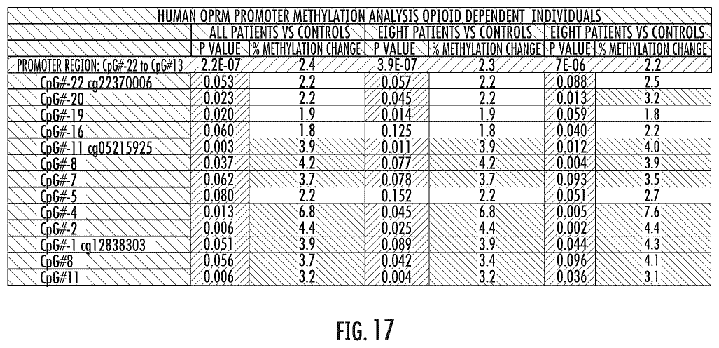

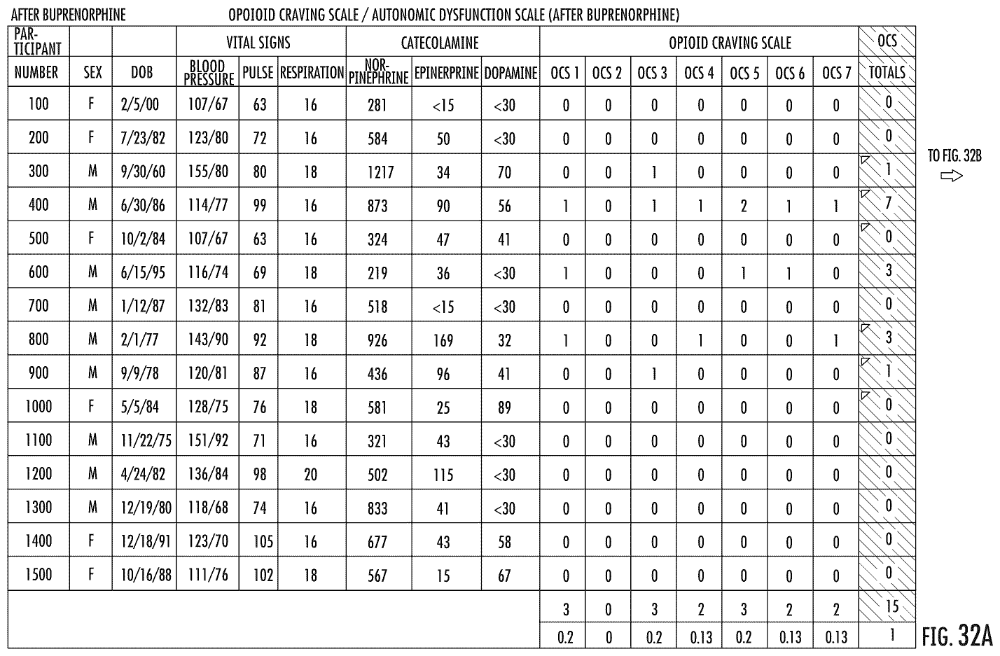

As illustrated in FIGS. 16A-B, during the period of autonomic dysfunction prior to buprenorphine administration, the average ADS was 42.5 out of a possible 56. The average OCS was 22.7 out of a possible 28. With reference to FIGS. 32A-B, two hours after administration of the buprenorphine 16 mg, the average ADS fell 88% to 4.9 out of a possible 56. The average OCS fell 96% to 1.0 out of a possible 28. The two subjects that had elevated norepinephrine and epinephrine combined had OCS scores of 26 (participant 800) and 26 (participant 1300), and ADS scores of 34 (participant 800) and 49 (participant 1300).

Using a two-sided t-test and pooled standard deviation, the differences in catecholamine level values between the general population and subjects in opioid withdrawal, as well as the differences between the general population and those stabilized on buprenorphine were evaluated. For the former comparison, at a significance level of 0.05, significant differences were found for dopamine (p=0.0194), epinephrine (p=0.0083), and norepinephrine values (p=0.0454). For the latter comparison, catecholamine level values were insignificant. Catecholamine levels below a certain value were difficult to detect and excluded from the analysis. For dopamine values, this was recorded as <30. For epinephrine values, this was recorded as <15. For analysis purposes, these values were replaced with 30 and 15, respectively.

It should be noted that catecholamine levels, while significantly improved, had not fully normalized two hours after the administration of the buprenorphine. Moreover, elevated catecholamine levels persisted even following buprenorphine induced stabilization in the subject.

### DISCUSSION

As discussed above, subjects who were in opioid withdrawal had elevated catecholamine levels. Significantly, the elevated catecholamine levels and the ADS/OCS scores were improved with a single dose of buprenorphine. To the inventor's knowledge, this is the first time elevated catecholamine levels, autonomic dysfunction, and opioid craving have been evaluated and shown to exist together. Both ADS and OCS were determined to be effective tools to measure autonomic dysfunction and opioid craving.

These findings call into question the accuracy of the default mental health diagnosis of Opioid Use Disorder (OUD). Based on the results of this study, the authors propose a more accurate diagnosis: autonomic dysfunction with catecholamine storm and associated opioid craving or the like. Millions of people have been denied buprenorphine treatment due to the misdiagnosis of OUD. This would explain why the opioid crisis and the epidemic of opioid overdose deaths have not been attenuated despite the ample resources brought to bear.

### Example 2: DNA Methylation

Following IRB approval and under informed consent, 8 of the 15 study participants from Example 1b (see above) submitted a saliva sample for DNA methylation analysis. Another 7 opioid dependent subjects from a treatment program also voluntarily provided saliva samples for DNA analysis. DNA methylation analysis was performed on 6 out of the 7 treatment program volunteer subjects. See Table 5, below.

TABLE 5

| Sample Group | Sample ID |
|---|---|
| Control | 7774 |
| Control | 6628 |
| Control | 6765 |
| Control | 6680 |
| Control | 5104 |
| Clinic | 6997 |
| Clinic | 7532 |
| Clinic | 6753 |
| Clinic | 7099 |
| Clinic | 6772 |
| Clinic | 6295 |
| Clinic | 7317 |
| Study | 7341 |
| Study | 6900 |
| Study | 5826 |
| Study | 7530 |
| Study | 7478 |
| Study | 29439 |
| Study | 7031 |
| Study | 7488 |

### Methylation Analysis Protocol

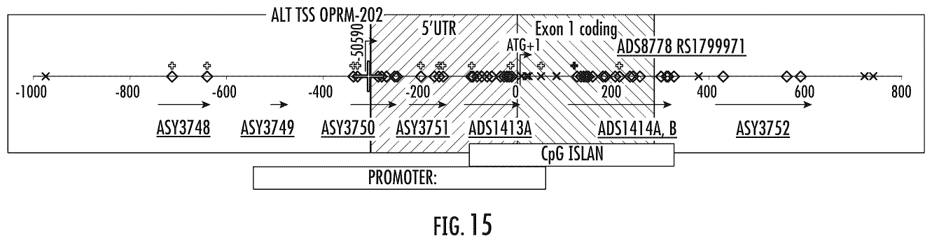

An analysis of the methylation percentages of specific CpG sites within the human OPRM1 gene, which encodes for the human opioid receptor mu 1, was performed by EpiGenDx, Inc. The human OPRM1 genomic DNA sequence is available at NCBI, Genbank, Accession No. AY587764 (https://www.ncbi.nlm.nih.gov/nuccore/AY587764.1). FIGS. 1, 4A-C, 7A-C, and 10A-C define the CpG sites that were targeted for methylation analysis within the OPRM1 gene, which includes sites both upstream and slightly downstream the OPRM1 transcription start codon. CpG sites within the CpG Island were also targeted for analysis as shown in FIG. 15.

CpG sites analyzed included previously characterized CpG sites cg22370006 (CpG #-22), cg14262937 (CpG #-21), cg06649410 (CpG #-15), cg23143142 (CpG #-13), cg23706388 (CpG #-12), cg05215925 (CpG #-11), cg14348757 (CpG #-10), cg12838303 (CpG #-1), cg22719623, and cg15085086. See Sandoval-Sierra et al., Effect of Short-term Prescription Opioids on DNA Methylation of the OPRM1 Promoter, Clinical Epigenetics (2020) 12:76, https://doi.org/10.1186/sI3148-020-00868-8; Chorbov et al., Elevated Levels of DNA Methylation at the OPRM1 Promoter in Blood and Sperm from Male Opioid Addicts, Opioid Manag. 2011; 7(4):258-264.

CpG sites were measured by Targeted Next-Gen Bisulfite Sequencing for DNA Methylation Analysis using in silico designs to amplify specific regions both upstream and downstream of the OPRM1 start codon (ATG). Each regulatory element of a requested gene was carefully evaluated before beginning the process of assay design. Gene sequences containing the target of interest were acquired from the Ensembl genome browser and annotated. The target sequences were re-evaluated against the UCSC genome browser for repeat sequences including LINE, SINE, and LTR elements. Sequences containing repetitive elements, low sequence complexity, high thymidine content, and high CpG density were excluded from the in silico design process.

DNA was extracted using the Agencourt DNAdvance™ Kit (Beckman Coulter; Brea, CA; cat #A48705) per the manufacturer's protocol with minor modification. The gDNA samples were eluted using DNA elution buffer in 50 μL. 500 ng of extracted DNA samples were bisulfite modified using the EZ-96 DNA Methylation-Direct Kit™ (ZymoResearch; Irvine, CA; cat #D5023) per the manufacturer's protocol with minor modification. The bisulfite modified DNA samples were eluted using M-elution buffer in 46 μL.

All bisulfite modified DNA samples were amplified using separate multiplex or simplex PCRs. PCRs included 0.5 units of HotStarTaq (Qiagen; Hilden, Germany; cat #203205), 0.2 μM primers, and 3 μL of bisulfite-treated DNA in a 20 μL reaction. All PCR products were verified using the Qiagen QIAxcel Advanced System (v1.0.6). Prior to library preparation, PCR products from the same sample were pooled and then purified using the QIAquick PCR Purification Kit columns or plates (cat #28106 or 28183). PCR cycle conditions were 95° C. 15 min; 45×(95° C. 30 s; Ta° C. 30 s; 68° C. 30 s); 68° C. 5 min; 4° C. ^{∞}.

Libraries were prepared using a custom Library Preparation method created by EpigenDx. Next, library molecules were purified using Agencourt AMPure XP beads (Beckman Coulter; Brea, CA; cat #A63882). Barcoded samples were then pooled in an equimolar fashion before template preparation and enrichment were performed on the Ion Chef™ system using Ion 520™ & Ion 530™ ExT Chef reagents (Thermo Fisher; Waltham, MA; cat #A30670). Following this, enriched, template-positive library molecules were sequenced on the Ion S5™ sequencer using an Ion 530™ sequencing chip (cat #A27764).

FASTQ files from the Ion Torrent S5 server were aligned to a local reference database using the open-source Bismark Bisulfite Read Mapper program (v0.12.2) with the Bowtie2 alignment algorithm (v2.2.3). Methylation levels were calculated in Bismark by dividing the number of methylated reads by the total number of reads.

### Results

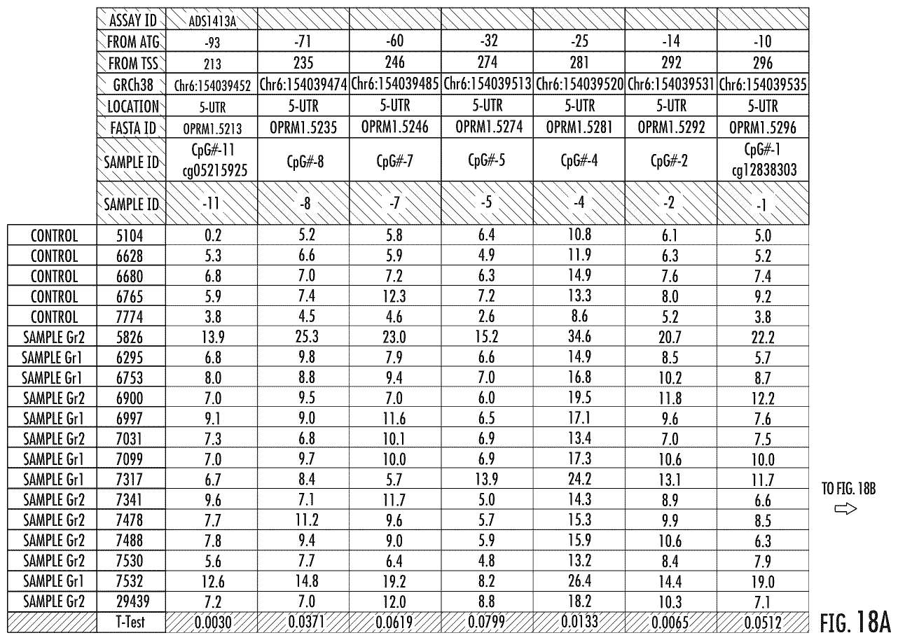

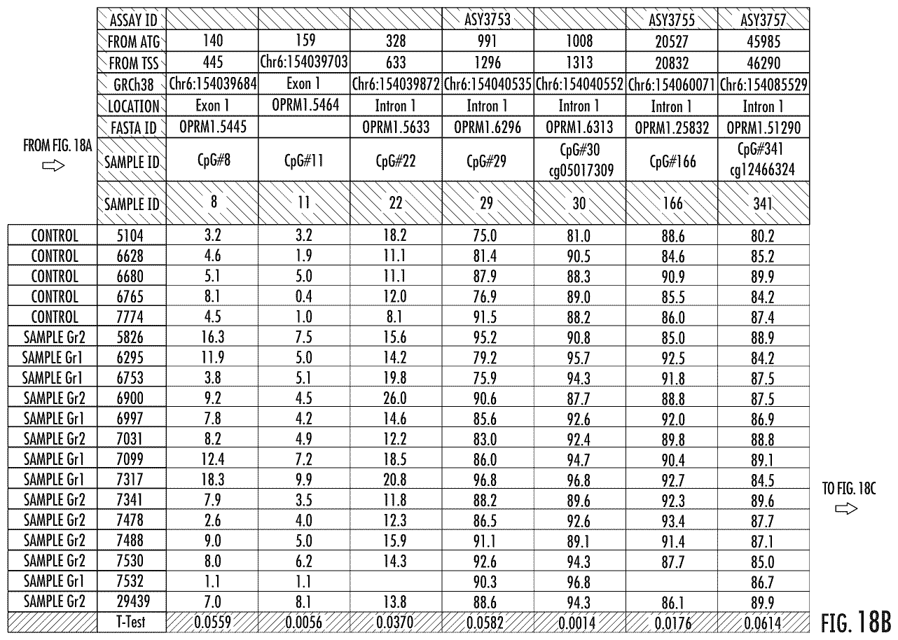

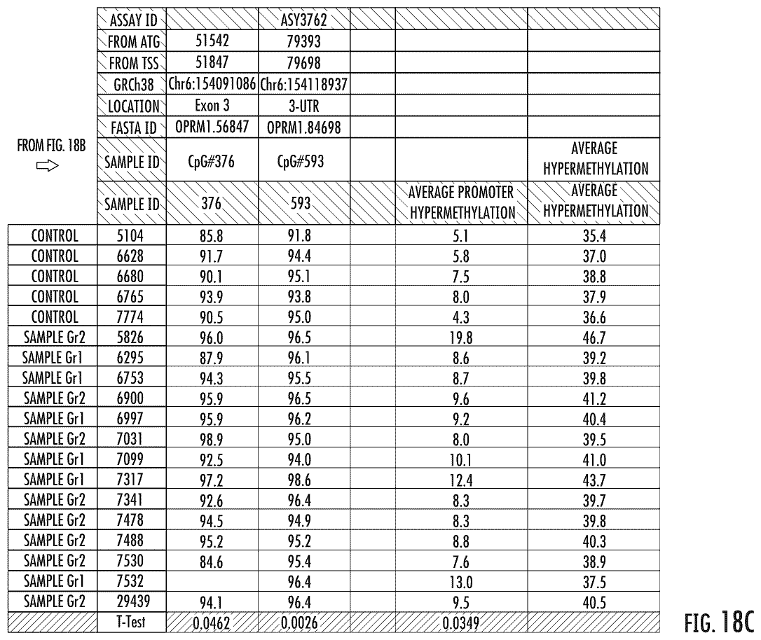

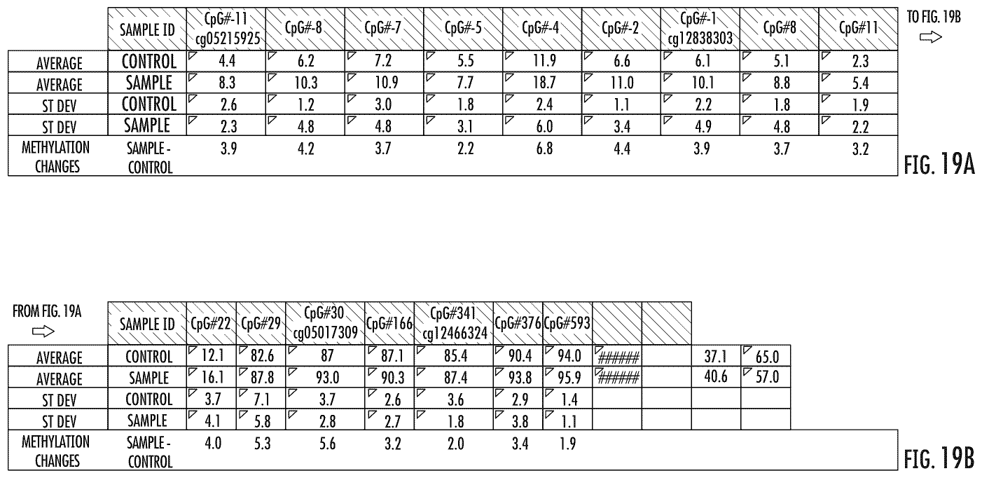

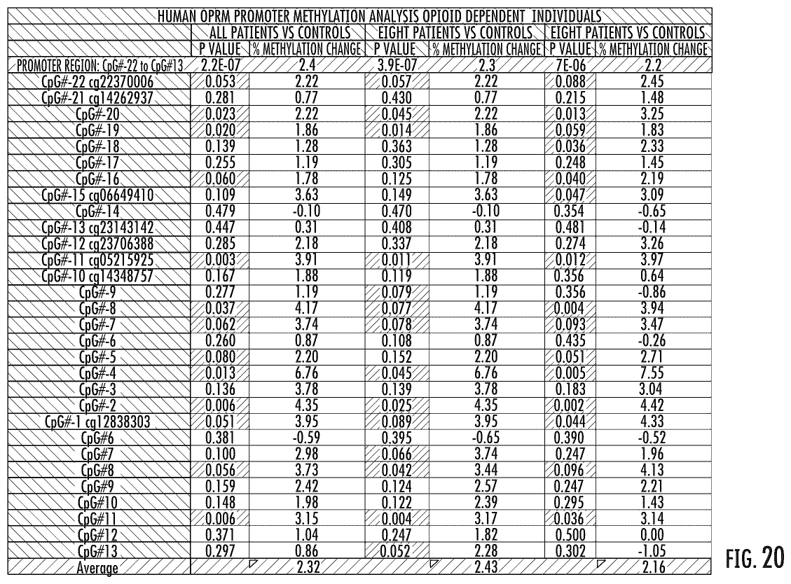

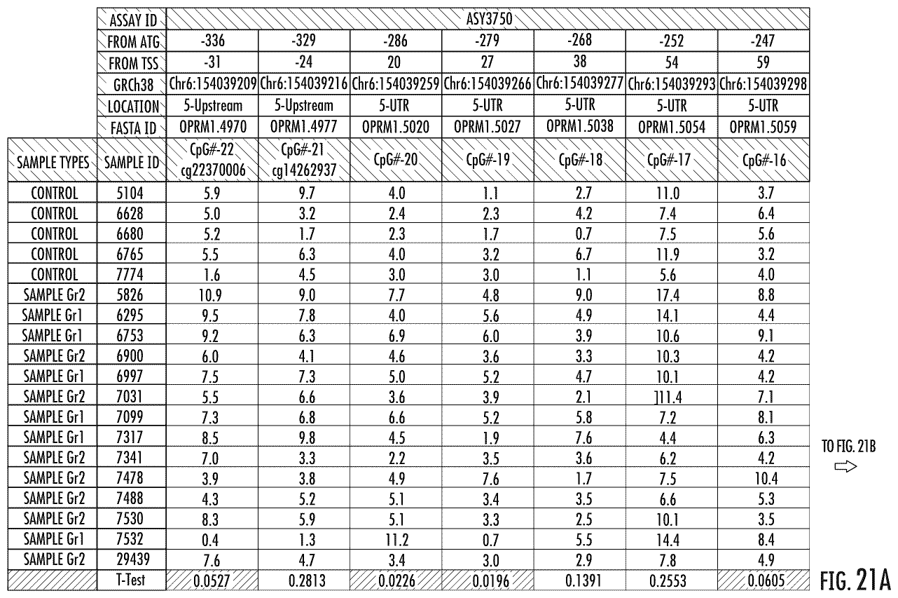

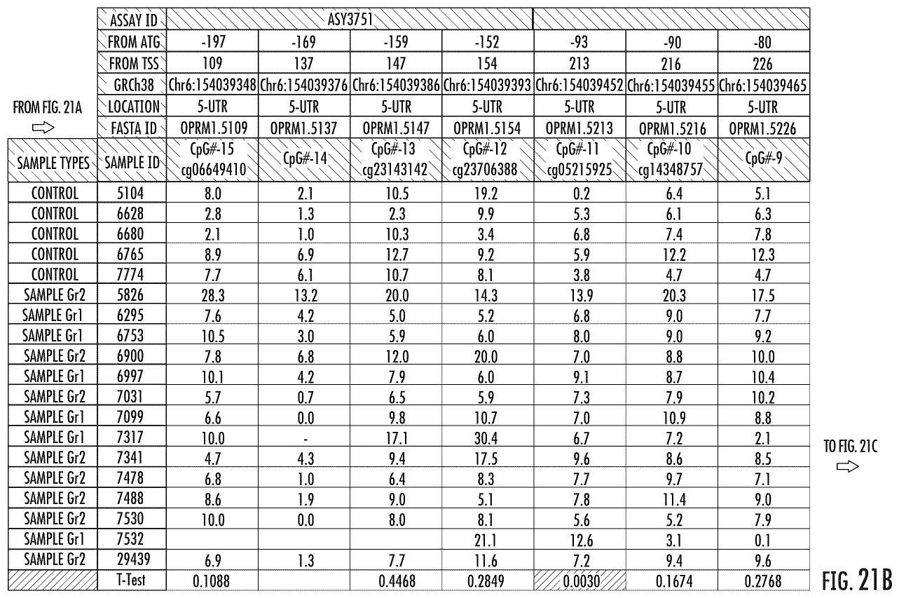

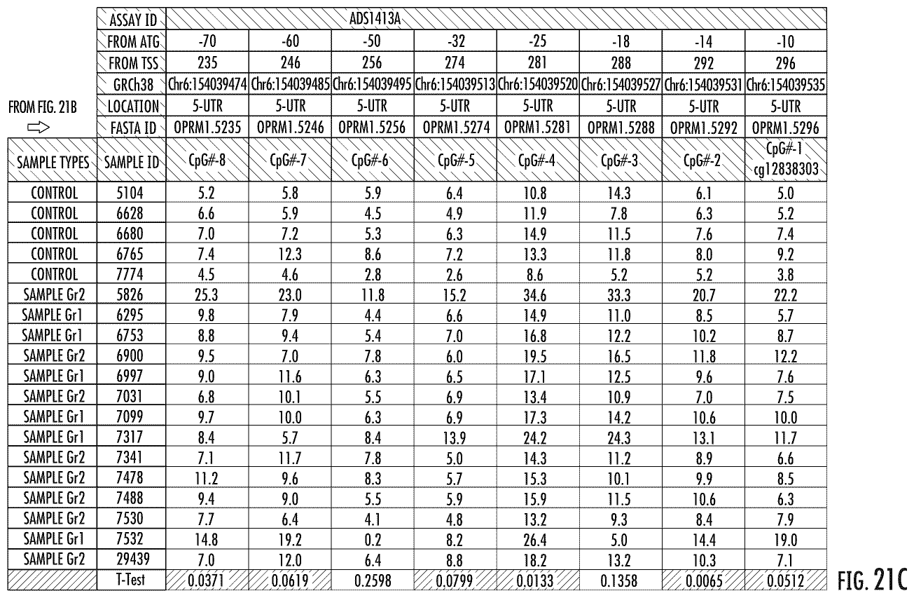

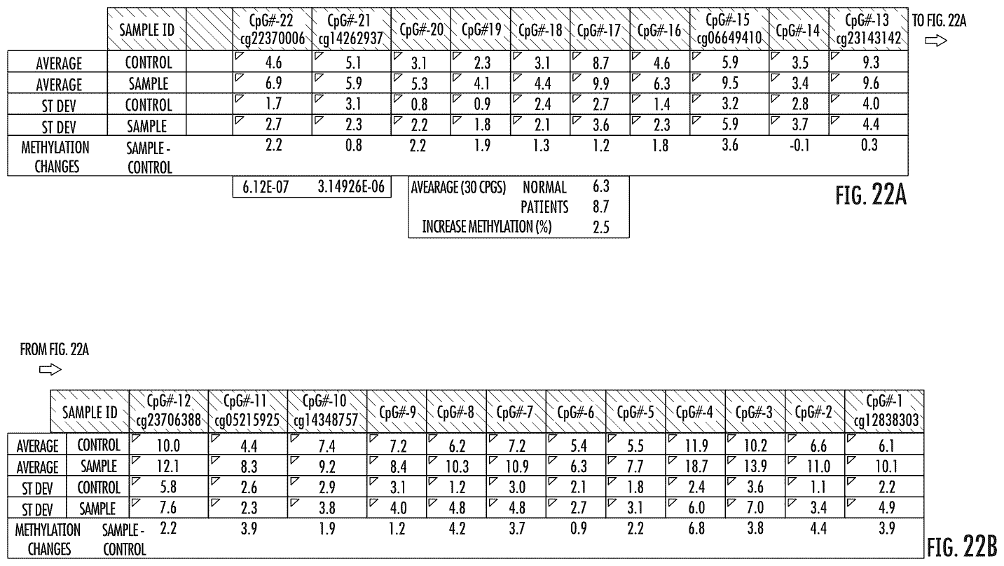

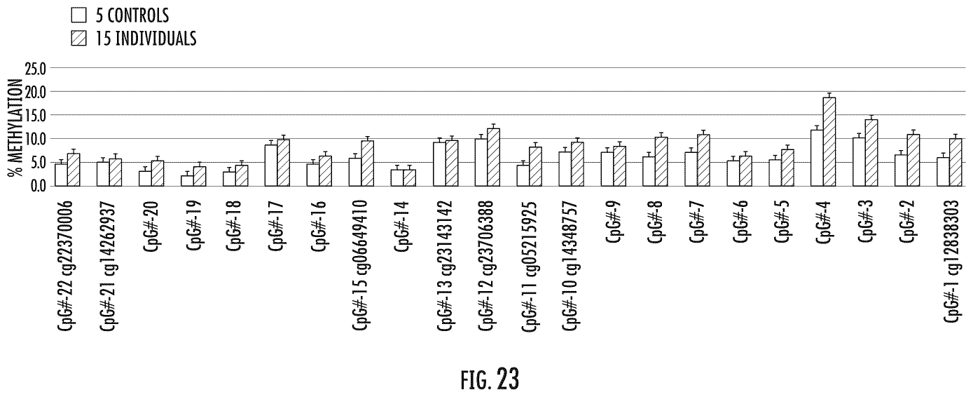

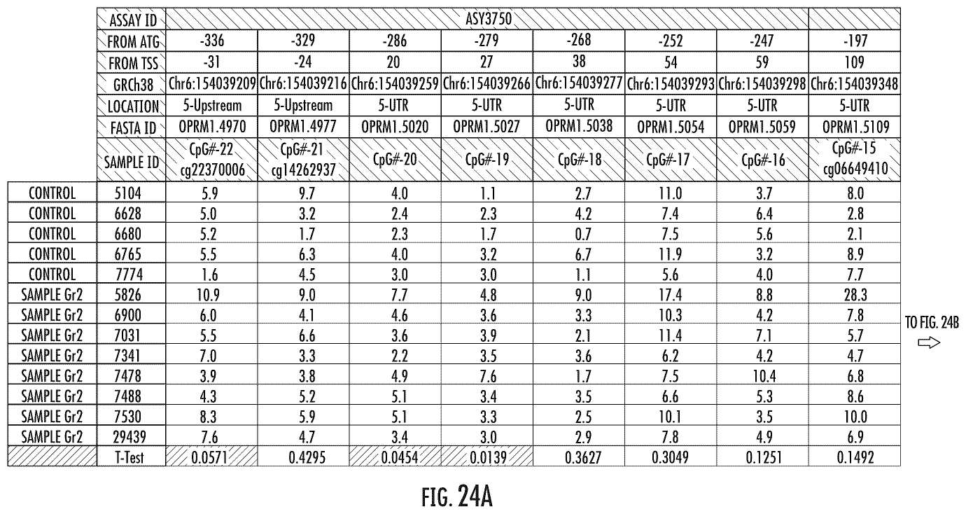

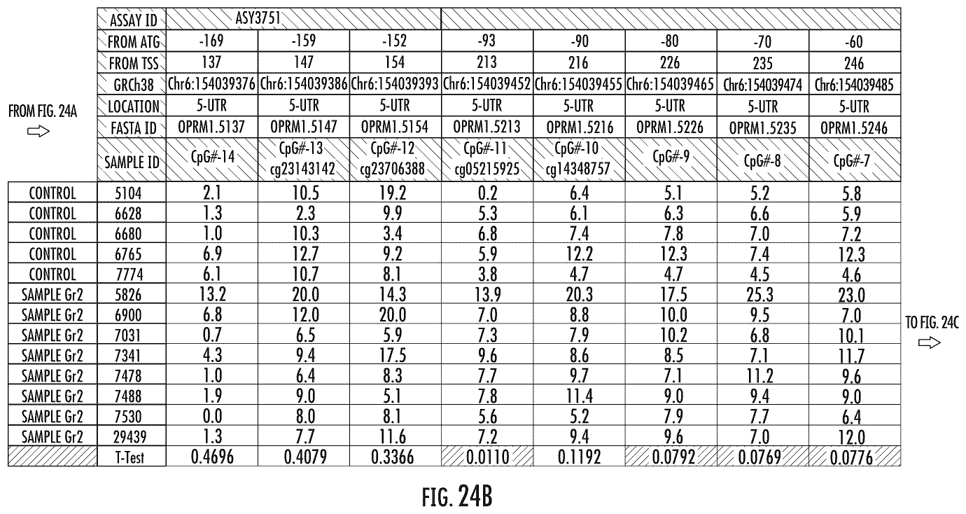

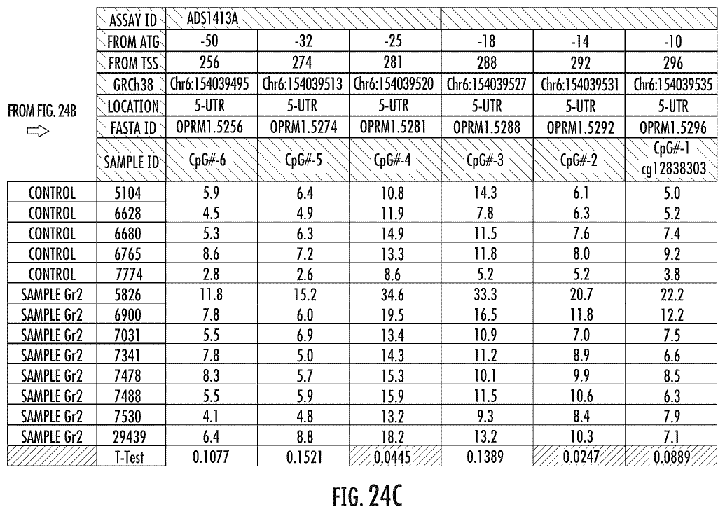

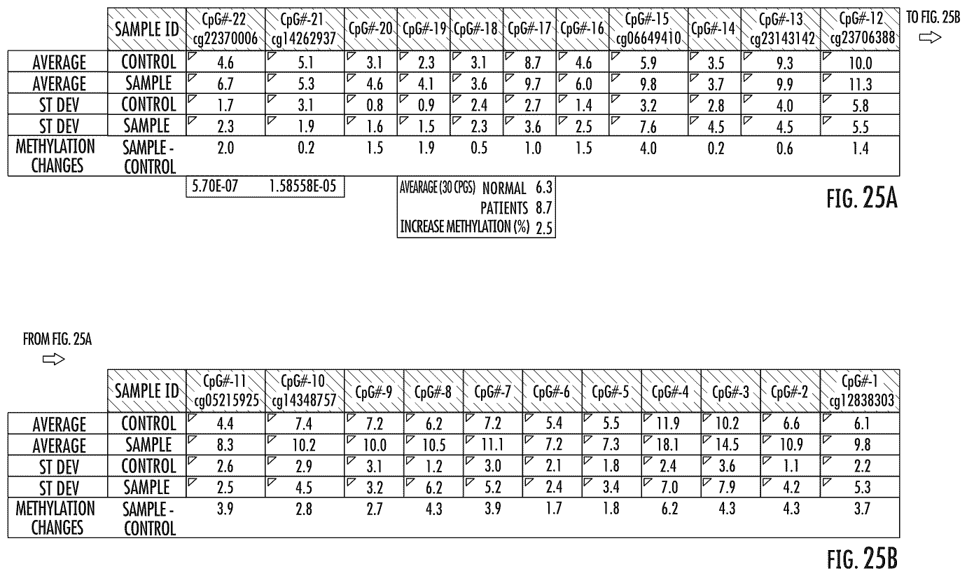

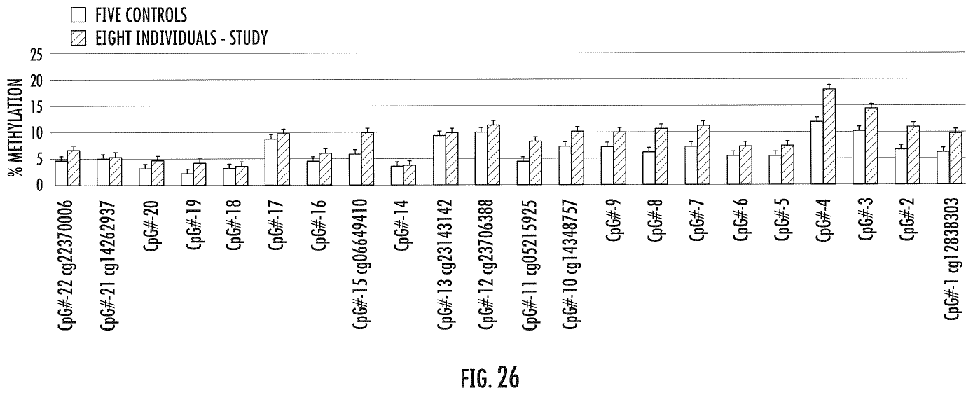

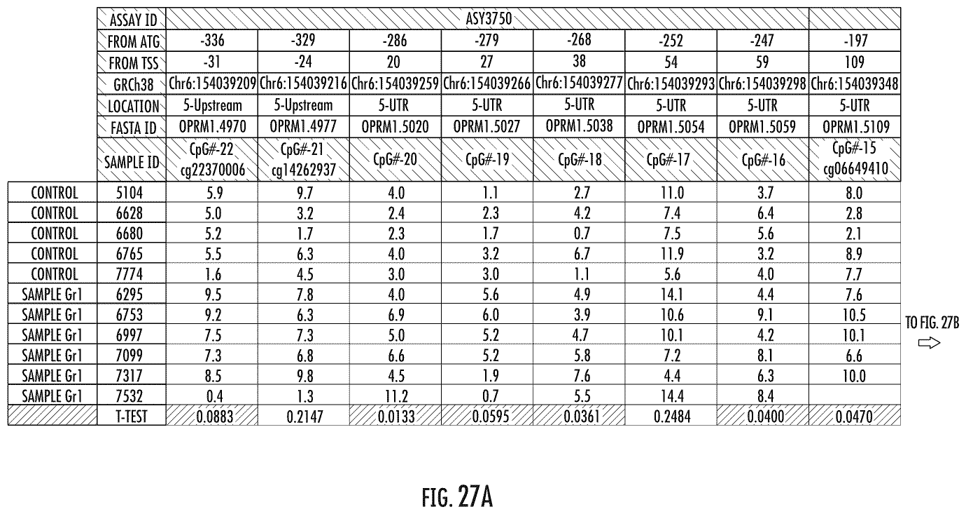

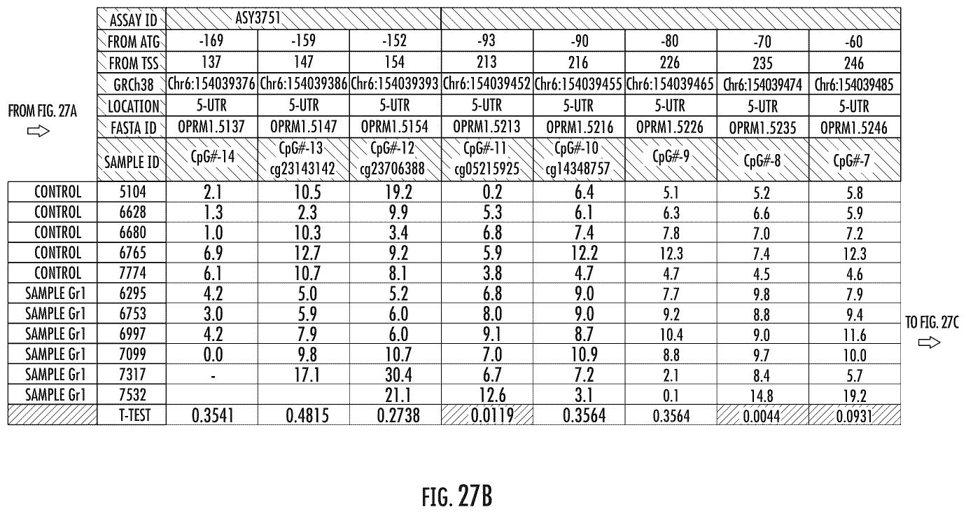

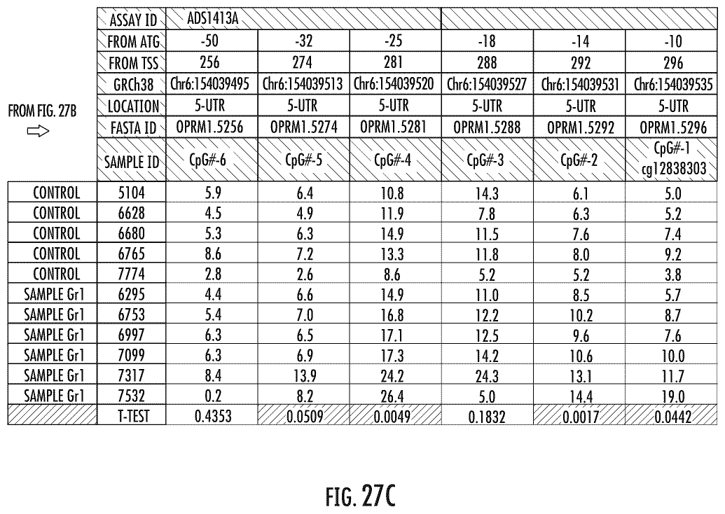

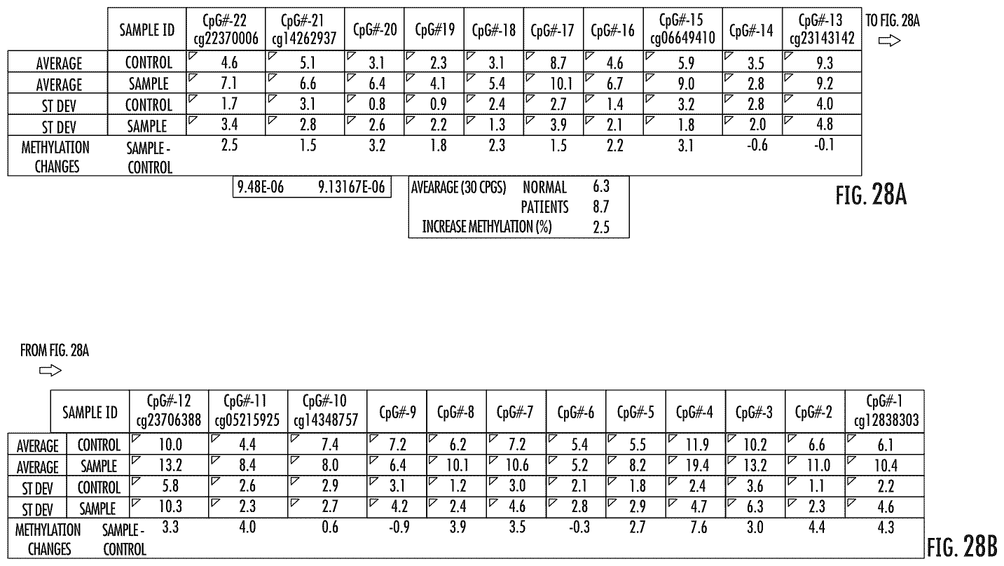

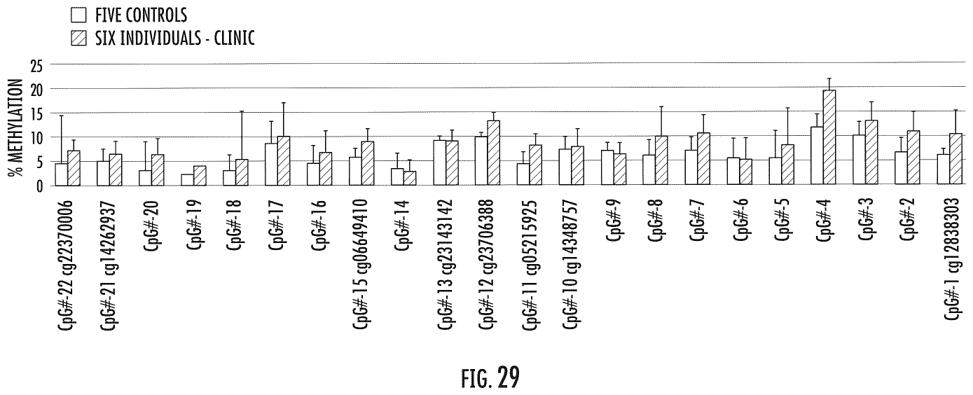

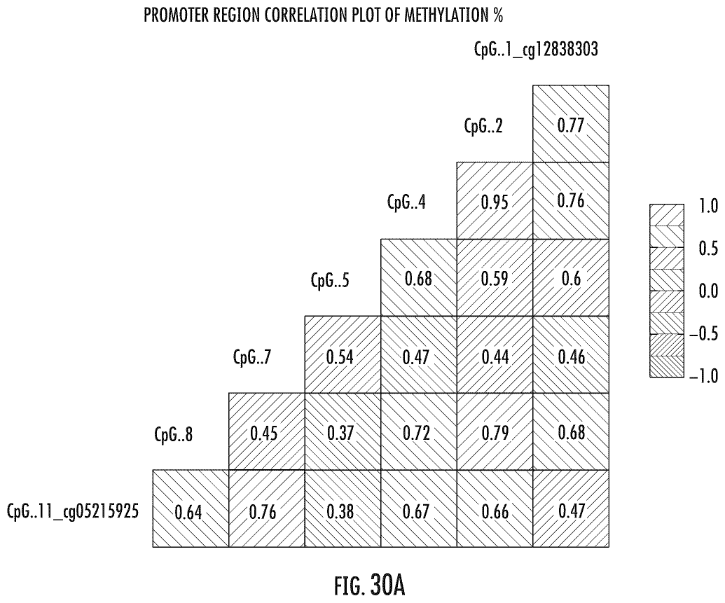

Referring to FIGS. 18 and 19, methylation was generally higher in test samples as compared to control samples. This phenomenon was especially pronounced for CpG sites positioned within the CpG Island just upstream and downstream of the ATG start codon e.g., between base positions −93 to 159. For example, with respect to CpG #−4 (at −25 from ATG), the average percent methylation was 11.9% (Standard Deviation=2.4) for control versus 18.7% (SD=6.0) for test samples—an increase of 6.8%. For CpG #−11 cg05215925 (at −93 from ATG), the average methylation percent was 4.4% for control (SD=2.6) and 8.3% for test samples (SD=2.3)—an increase of 3.9%. For CPG #−2 (at −14 from ATG), the average methylation percent was 6.6% for control (SD=1.1) versus 11.0% for test samples (SD=3.4)—an increase of 4.4%. CpG sites downstream of position+328 also showed methylation increases, but they were less dramatic.

Referring to FIGS. 16A-C, 17, and 20 the methylation percent increases for the following CpG sites demonstrated p-values of <0.1 (average percent change and average p value test subjects v. controls)—CpG #-22 cg 223700006 (2.3% average increase/p value=0.066), CpG #-20*(2.5% average increase/p value=0.027), CpG #-19*(1.86% average increase/p value=0.031), CpG #-16*(1.93% average increase/p value=0.075), CpG #-11 cg05215925 (3.9% average increase/p value=0.008), CpG #-8*(4.1% average increase/p value 0.039), CpG #-7*(3.63% average increase/p value=0.077), CpG #-5*(2.36% average increase/p value=0.094), CpG #-4*(7.07% average increase/p value 0.021), CpG #-2*(4.4% average increase/p value=0.011), CpG #-1 cg12838303 (4.03% average increase/p value=0.061), CpG #8*(3.73% average increase/p value=0.064), and CpG #11*(3.16% average increase/p value=0.015). Among the 13 with p values <0.1, ten CpG sites (denoted with an *) had not been previously associated with hypermethylation and six out of those ten had p values <0.05. Other (3′ of start codon) CpG sites located further downstream (3′ of the start codon/ATG) demonstrated hypermethylation and positive p-values (<0.1) include CpG #13, CpG #15, CpG #22, CpG #29, CpG #30, CpG #35, CpG #38, CpG #166, CpG #341, CpG #376, CpG #593, and CpG #594. See FIG. 16A-C.

### Statistical Analysis and Diagnostics Using Artificial Intelligence

Statistical analyses were performed using R (R version 4.2.1), the program for statistical computing. Descriptive statistics of the DNA methylation data of CpG sites was summarized, comparatively grouped by the Control and Experimental groups. All boxplots, density plots, bar plots, and heatmaps were created using ggplot2 (version 3.3.6). All tables were produced by “kable” in the kableExtra package (version 1.3.4). Assumptions for statistical methods were checked with the “mvn” function in the MVN package (version 5.9), and “levene test” function for equal covariance from rstatix.

a. Spearman's Rank-Based Correlations

A Spearman's rank-based correlation analysis was performed comparing each CpG site were explored, connecting those relationships with the Promoter and non-Promoter regions of the OPRM1 gene. Correlations and visualizations were constructed by the “ggcorr” function in GGally (version 2.1.2). Principal Component Analysis (PCA) and Non-Metric Multidimensional Scaling (nMDS) were implemented to visualize group separation of the samples. nMDS was built using the “metaMDS” function in the vegan package (version 2.6-2) specifying for Euclidean distance, and PCA used the “prcomp” function in base stats package. Biplots and scree plots were produced from the “fviz” function in factoextra package (version 1.0.7), as well as ggplot2.

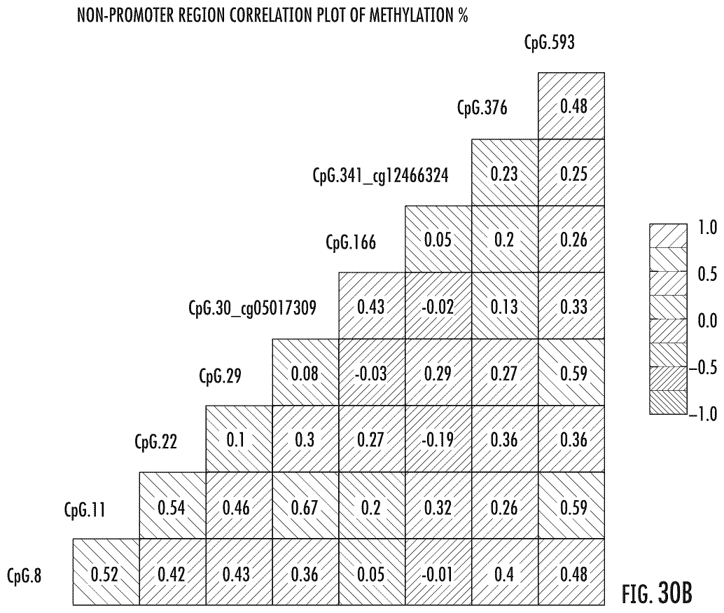

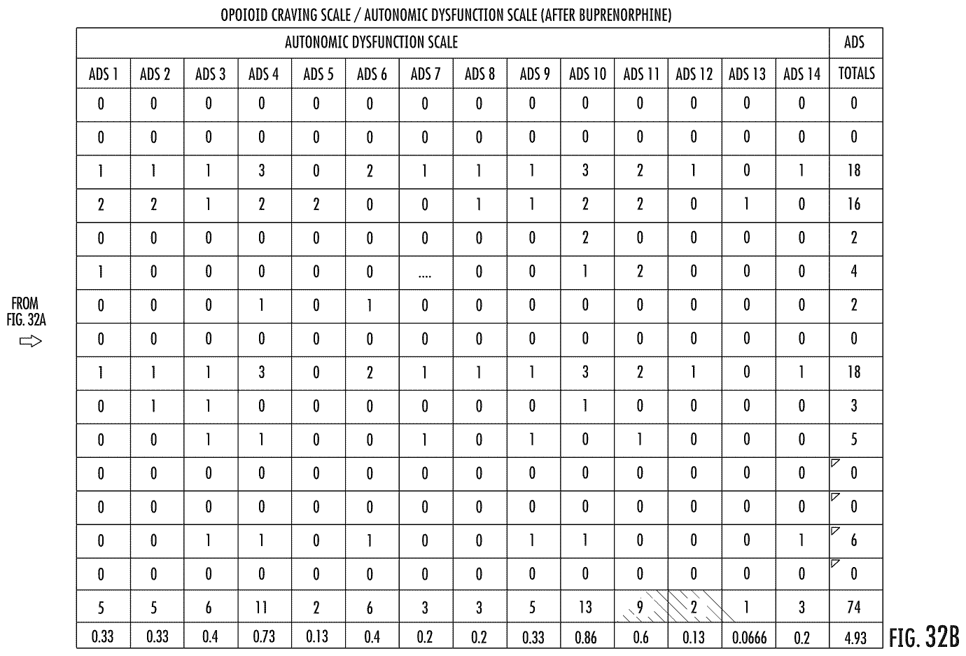

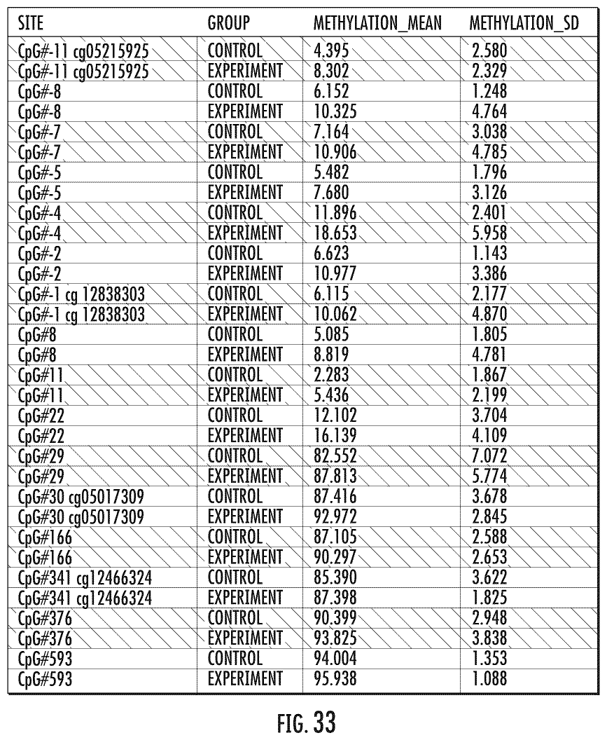

The Correlation Plot of Methylation shown in FIG. 30A-B compares collinearity between seven CpG sites within the promoter region (FIG. 30A) and nine CpG sites outside the promoter region (FIG. 30B) of the human OPRM-1 gene. Note that CpG #8, CpG #11, and CpG #22 may overlap or fall within the promoter region. Generally, a stronger collinearity was found among the CpG sites within the promoter region. Mean methylation percentages and standard deviations for the sixteen CpG sites are reflected in FIG. 33. For example, referring to FIG. 30A, a percent positive correlation (>0.5) was found between, for example, CpG #-5/CpG #-4, CpG #5/CpG #-2, CpG #-5/CpG #-1, CpG #-7/CpG #-5, and CpG #-8/CpG #-1 (cg12838383). A strong positive correlation (>0.7) was found between, for example, CPG #-11(cg05215925)/CPG #-1(cg12838383), CpG #-4/CPG #-1(cg12838383), CpG #-8/CpG #-4, and CpG #-11(cg05215925)/CpG #-8. A very strong positive correlation (>0.9) was found between, for example, CpG #-4/CpG #-2. While some collinearity exists, the non-promoter region CpG sites were generally weaker. See FIG. 30B.

This strong collinearity provides evidence that unsupervised machine learning such as Principal Component Analysis (PCA) could prove to be useful in differentiating the two populations: the opioid naïve and the opioid dependent. Principal Component Analysis is described in more detail below.

b. Principal Component Analysis (PCA) and Non-Metric Multidimensional Scaling

Principal Component Analysis (PCA) and Non-Metric Multidimensional Scaling (NMDS) were implemented to visualize group separation of the samples. NMDS was built using the “metaMDS” function in the vegan package (version 2.6-2) specifying for Euclidean distance, and PCA used the “prcomp” function in base stats package. Biplots and scree plots were produced from the “fviz” function in factoextra package (version 1.0.7), as well as ggplot2.

i. Principal Component Analysis (PCA)

The 16 CpG sites discussed above were subjected to Principal Component Analysis (PCA), a widely used form of unsupervised machine learning and a dimension reduction technique particularly useful in the setting of a large quantity of variables and a small sample size. If Principal Component Analysis can visually differentiate the two groups (naïve control vs. opioid dependent/abstaining), then it could be a powerful diagnostic tool to show the epidemiological uniqueness of the genetically damaged population with potential DNA based clinical test applications. If Principal Component Analysis cannot recognize the two genetic populations as distinct, then this would be supportive of the mental health diagnosis of Opioid Use Disorder.

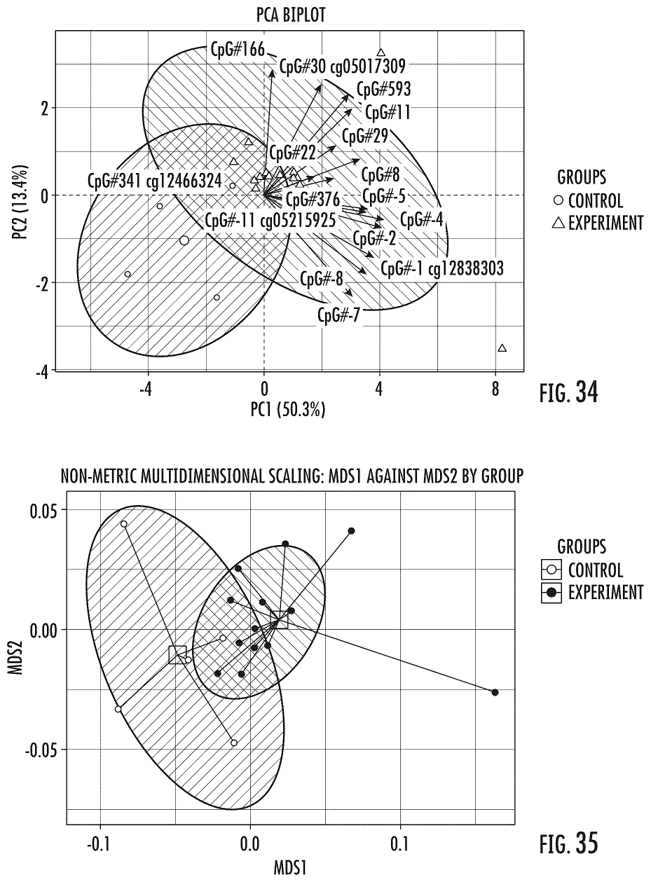

FIG. 34 is a biplot graph prepared from the Principal Component Analysis data using points and vectors to represent structure. The Principal Component scores are the points and the loading of the samples are the vectors. The biplot shown in FIG. 34 plots PC1 against PC2 and highlights the separation between the Control Group (opioid naive) and the Experiment Group (opioid dependent) created by the first two PCs. Overall, the Control Group is plotted in the bottom left quadrant indicating that the Controls have PC1 and PC2 values that are smaller and negative. However, the Experiment Group are primarily grouped from the middle to the right, meaning that the Experiment Group has higher values than the Control Group. The fact that the machine sees the two genetic populations as separate and distinct is evidence supporting the diagnosis of autonomic dysfunction due to genetic damage and evidence undermining the diagnosis of Opioid Use Disorder.

ii. Non-Metric Multidimensional Scaling

Like Principal Component Analysis (PCA), Non-Metric Dimensional Reduction Scaling (NMDS) is a dimensional reduction technique. NMDS optimizes stress, which are values equivalent to the difference in distance between the reduced dimension and the full dimension (the original data). Optimizing the stress values means that the algorithm will try to minimize the stress and therefore maximize the similarities between the reduced and full dimensions. NMDS differs from PCA as NMDS relies upon these calculated stress values for orientation. PCA relies upon the loadings calculated by eigenvalues/eigenvectors. FIG. 35 is the NMDS plot of MDS1 against MDS2. The distances calculated are measured in Euclidean. The data was transformed using Hellinger's square root method. NMDS and PCA outcomes were compared.

NMDS produced a similar plot/values as PCA, but with a slightly different spread. The Control Group is again predominantly on the negative side of the x-axis and the Experimental Group is again predominantly on the middle/positive side of the x-axis. While outliers are acknowledged (possibly attributable to small sample size), the NMDS results corroborate the PCA analysis results. Opioid naïve and opioid dependent subjects are genetically distinguishable with DNA methylation of the OPRM1 gene.

c. P-value Correction (Mann-Whitney, Kruskal-Wallace, Anova Tests)

The analysis of DNA methylation percentages of the OPRM gene were evaluated at a CpG site basis, stratified by the two groups of interest: Control and Experiment. Multiple tests for comparison of the groups were analyzed using the non-parametric Wilcox-Mann-Whitney (WMW) U-test, also known as the Wilcox Rank Sum Test. The WMW U-test was performed using wilcox test function in the rstatix package (version 0.7.0), specifying the test as a one-sided, unpaired test. The 16 tests (for the 16 CpG sites) were corrected using the Benjamini-Hochberg correction. Additionally, the Kruskal-Wallace test and one-way ANOVA test were implemented as alternatives to the WMW U-test, both from the rstatix package.

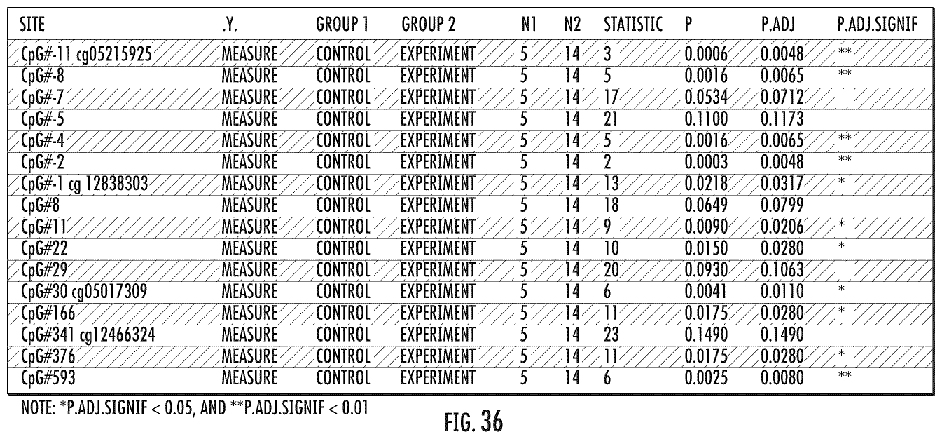

The Mann-Whitney U-Test is a specific version of the Kruskal-Wallace Test intended for exactly two groups. The Null Hypothesis is that the two groups come from the same population. As shown in FIG. 36, using alpha=0.05 and correcting the p-values, 11 of the 16 CpG sites (bolded) are significantly different between the Control (opioid naive) and Experimental (opioid dependent) groups.

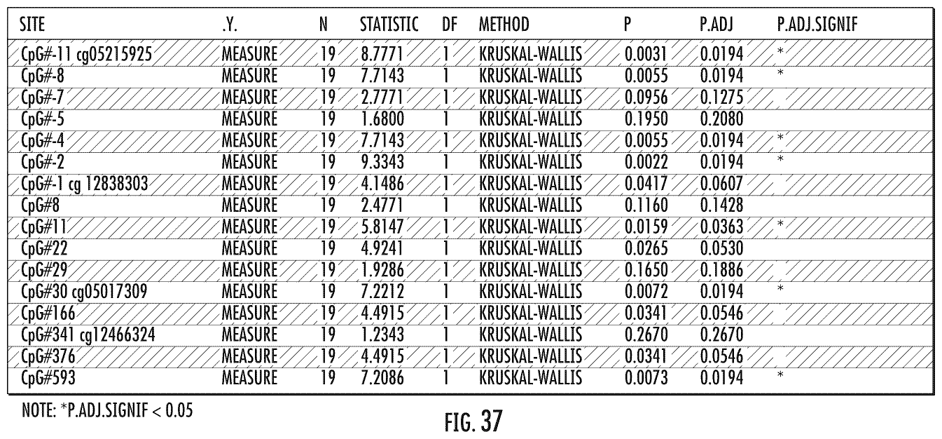

The Kruskal-Wallace Test is the non-parametric alternative to a one-way ANOVA (Analysis of Variance) and is a broader version of the Mann-Whitney U-Test. Instead of testing group means, the Kruskal-Wallace Test analyzes for significant differences between the mean ranks of the groups. The Null Hypothesis is that the two groups come from the same population. As can be seen in FIG. 37, using alpha=0.05 and correcting the p-values, 7 of the 16 CpG sites (bolded) are significantly different between the Control (opioid naive) and the Experimental (opioid dependent) groups.

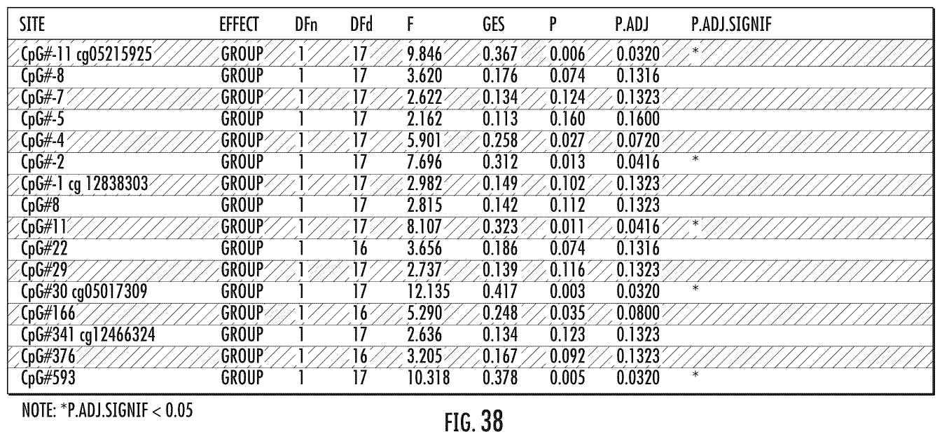

The one-way ANOVA Test is a parametric approach to compare two or more groups. The Null Hypothesis of the ANOVA Test is that there is no difference among group means. As can be seen in FIG. 38, using alpha=0.05, and correcting the p-values, 5 of the 16 CpG sites (bolded) are significantly different between the Control (opioid naive) and the Experimental (opioid dependent) groups.

d. Permanova Analysis (Permutational Multivariate Analysis of Variance)

A PERMANOVA model (non-parametric) was built using the “adonis2” function in the vegan package (version 2.6-2) to investigate the multivariate relationship of the CpG sites by group. Measures of dissimilarity, or distances, were calculated using Euclidean distances.

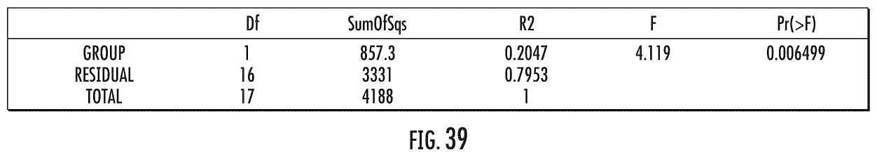

PERMANOVA is used to test whether groups of objects are significantly different. The test statistic is the pseudo-F-ratio. The Null Hypothesis for the PERMANOVA is that the centroids and dispersions of the groups as defined by measured space are equivalent for all groups. The PERMANOVA model for this analysis was created using the 16 CpG sites as the dependent variables and Group as the independent variable. As can be seen in FIG. 39 using alpha=0.05, and with P<0.0065, the Null Hypothesis should be rejected meaning there is some difference between the centroids/dispersions of each group. This finding supports the group separation shown via PCA and NMDS.

### Treatment Regimen

Suitable treatment regimens, include, for example, administering a proper dosage of opioid agonist or partial agonist, such as buprenorphine to a subject who has been determined to have one or more of the following conditions: elevated catecholamine levels and/or elevated methylation in the promoter region of the human OPRM1 gene. In other embodiments, determining whether to administer treatment includes determining whether the test subject ADS and/or OCS scores are elevated in combination with the above.

Once subjects have been determined to be suffering from autonomic dysfunction, subjects may be administered buprenorphine on a bi-daily, daily, every two days, a bi-weekly basis, a weekly basis, a bi-monthly basis, and/or a monthly basis to be determined by qualified medical professional. In addition to using buprenorphine on some predetermined basis, the subject will initially be evaluated. Moreover, monitoring of the subject will occur on an ongoing basis. This monitoring will be specifically driven towards the end point of a reduction in the level of autonomic dysfunction. In certain embodiments of the invention, the monitoring of the subject can occur periodically on at least a daily basis, at least a weekly basis, at least a biweekly basis, at least a monthly basis, and any other periodic basis that is appropriate for the treatment of the subject. In other embodiments of the invention, the monitoring may be on a real-time basis. In still other embodiments of the invention, monitoring the subject may be remotely. As used herein, remotely is intended to mean not in the direct presence of the medical personal and/or the system that provides analysis of the results.

Abnormal levels of autonomic activity can be detected, measured, and monitored in a variety of ways, for example, via direct detection and monitoring of the activity of branches of the sympathetic nervous system—both sympathetic and parasympathetic. In certain embodiments of the invention, the monitoring can include direct detection and monitoring of laboratory values (e.g., blood catecholamine concentrations) in a plurality of bodily tissues and fluids. In other embodiments of the invention, the monitoring includes indirect monitoring of the autonomic nervous system by such methodologies as monitoring the heart rate variability and through a plurality of equipment. In certain other embodiments of the invention, monitoring is intended to cover all types of sympathetic nervous system monitoring and for the ideal outcome of each subject. Upon information and belief, left untreated, autonomic dysfunction can be a risk factor for the development of opioid induced adrenal insufficiency.

While direct measurement of nerve activity and measurement of lab values have both been part of the medical landscape for some time, utilizing equipment in the home and to monitor the status of the autonomic nervous system is a novel concept in and of itself. For example, according to certain embodiments of the invention, there are a variety of ways to determine heart rate variability. The invention intends to encompass a multiplicity of methodologies for the measurement of heart rate variability when the results are utilized to assess the status of the autonomic nervous system. According to an embodiment of the invention, the use of a smart phone and a technology known as photoplethysmography (PPG) may be used to determine heart rate variability. In PPG, the workings of the smart phone camera are utilized to detect both transmission through and reflection from the body tissue. Based upon the level of blood perfusion, heart related information can be obtained. From the data collected, a heart rate variability can be calculated. Good heart rate variability is associated with good autonomic nervous system function. The lack of variability in heart rate is associated with abnormal function within the autonomic nervous system. The data may be either uploaded through an App or an Artificial Intelligence (“AI”) device, and logic can then be applied to the data.

In an embodiment of the invention, from this process, predetermined recommendations for treatment can be made. Further pursuant to this embodiment of the invention, both ongoing treatment with buprenorphine and a possible tapering off of the amount of buprenorphine to be used can be accomplished. This is vastly superior over any other known current process for monitoring and tapering of a replacement drug therapy. It is but one more example of how the invention advances the art and improves the quality of life for a large number of people suffering from autonomic dysfunction.

In an embodiment of the invention, monitoring of the autonomic nervous system is used to provide additional guidance regarding, for example, other components of the subject's comprehensive treatment regimen e.g., buprenorphine. In certain embodiments of the invention, direct measurement of autonomic nerve activity is used for monitoring. In other embodiments of the invention, laboratory evaluation of tissue or bodily fluids are used for monitoring. In still other embodiments of the invention, indirect methods are used for monitoring. Further pursuant to this embodiment of the invention, heart rate variability may be used in the indirect monitoring of the subject.

According to an aspect of the invention, treatment with buprenorphine and remote real time subject monitoring will dramatically impact the ability to rescue and save the individuals caught up with the opioids. Through advancements in technology, through experience, and with the machine learning capabilities of Artificial Intelligence, utilizing data collected from the subject in real time and during a subject's normal daily activities, enables the subject to be monitored and advised on health issues. According to an embodiment of the invention, these technologies are important in advancing the human life expectancy. One of the first roles of this tool will be in ending the opioid crisis.

The methods of treatment of the invention may also be applied to subjects suffering opioid induced autonomic dysfunction due to genetic damage whether such damage is now known or later discovered. In an embodiment of the invention, the types of genetic damages may be due to the genetic sequence of the nucleotides, known Epigenetic changes, or Epigenetic changes yet to be determined.

In certain embodiments of the invention, combination treatment with the medication and remote monitoring of the autonomic dysfunction. In certain other embodiments of the invention, the data along with artificial intelligence is stored in a cloud arrangement. The invention represents a major step forward in the care and treatment of millions of people suffering from autonomic dysfunction. The invention is expected dramatically and permanently to decrease the death rate from opioid overdose.

In another aspect of the invention, the autonomic nervous system is monitored for the purpose of providing guidance in tapering the amount of buprenorphine used in the treatment of the subject. Any and all methods for such monitoring as further described herein may be used in this assessment of the subject.

In other aspects, the invention can be described as follows:
(1)—A method for treating a subject suffering from autonomic dysfunction comprising:
obtaining a biological sample from a subject;
assaying the biological sample to determine catecholamine levels of one or more target catecholamines;
comparing said one or more target catecholamine levels to a reference catecholamine value;
determining, based on the comparison of the target catecholamine level to the reference catecholamine value, that one of more target catecholamine levels is greater than the reference catecholamine value; and
administering a treatment regimen comprising a predetermined dose of a partial opioid agonist to treat the subject when one or more target catecholamine levels is greater than the reference catecholamine value.
(2)—The method of claim 1, wherein the one or more target catecholamines is selected from the group consisting of epinephrine and norepinephrine.
(3)—The method of claim 1, wherein said reference catecholamine value comprises a normal concentration range of said target catecholamine.
(4)—The method of claim 1, wherein the partial opioid agonist is buprenorphine.
(5)—The method of claim 4, wherein said predetermined dose of buprenorphine is 16 mg.
(6)—The method of claim 1, wherein the biological sample is blood.
(7)—The method of claim 1, further comprising the steps of:
conducting a DNA methylation assay on the biological sample to determine DNA methylation levels in the subject;
comparing the DNA methylation levels to a reference DNA methylation value;
determining, based on the comparison of DNA methylation levels to a reference DNA methylation value, that the subject has elevated DNA methylation levels; and
administering a treatment regimen comprising a predetermined dose of a partial opioid agonist to treat the subject when said one or more target catecholamine levels is greater than the reference catecholamine value and/or when the subject has elevated DNA methylation levels as compared to the reference DNA methylation value.
(8)—The method of claim 7, wherein said reference DNA methylation value to determine if said subject has elevated DNA methylation comprises a normal control sample.
(9)—The method of claim 1, further comprising the steps of:
administering one or more surveys to the subject selected from Autonomic Dysfunction Scale and/or Opioid Craving Scale prior to treatment and recording a score for said one or more surveys;
comparing said scores from one or more surveys to a survey reference value; and
administering a treatment regimen comprising a predetermined dose of a partial opioid agonist to treat the subject if (1) said one or more target catecholamine levels is greater than the reference catecholamine value; and/or (2) said score from one or more surveys is above the reference survey value.
(10)—The method of claim 7, further comprising the steps of:
administering one or more surveys to the subject selected from Autonomic Dysfunction Scale and/or Opioid Craving Scale prior to treatment and recording a score for said one or more surveys;
comparing said scores from one or more surveys to a survey reference value; and
administering a treatment regimen comprising predetermined dose of a partial opioid agonist to treat the subject when (1) said one or more target catecholamine levels is greater than the reference catecholamine value; (2) said subject has elevated DNA methylation levels compared to the reference DNA methylation value; and/or (3) said score from one or more surveys is above the reference survey value.
(11)—The method of claim 7, wherein the partial opioid agonist is buprenorphine.
(12)—The method of claim 9, wherein said predetermined dose of buprenorphine is 16 mg.
(13)—The method of claim 7, wherein the biological sample is saliva.
(14)—The method of claims 7 and 10 wherein said DNA methylation assay is performed on the human OPRM1 promoter region.
(15)—The method of claims 7 and 10 wherein said DNA methylation assay is performed on the CpG Island region of the human OPRM1 promoter.
(16)—The method of claims 7 and 10 wherein said DNA methylation assay is performed on a plurality of specific CpG sites within the human OPRM1 promoter region.
(17)—The method of claims 7, 10, and 16 wherein said subject has elevated DNA methylation levels when at least one of the CpG sites in FIG. 33 has elevated methylation compared to the reference DNA methylation value.
(18)—The method of claims 7, 10, and 16 wherein said method further comprises the step of performing Principal Component Analysis (PCA) and/or Non-Metric Dimensional Reduction Scaling (NMDS) to confirm the results of the DNA methylation assay.
(18)—A method for treating a subject suffering from autonomic dysfunction comprising:
a. obtaining a biological sample from a subject;
b. assaying the biological sample to determine catecholamine levels of one or more target catecholamines; and
c. comparing said one or more target catecholamine levels to a reference value to determine if said subject has an elevated level of one or more target catecholamines; and/or
d. conducting a DNA methylation assay on the biological sample; and
e. comparing the results of said DNA methylation assay to a reference value to determine if said subject has elevated DNA methylation; and
f. administering a treatment regimen comprising a predetermined dose of a partial opioid agonist to treat the subject if said one or more target catecholamine levels is greater than the reference value or if the subject has elevated DNA methylation.
(19)—The method of claim 18 further comprising the steps:
administering one or more surveys to the subject selected from Autonomic Dysfunction Scale and/or Opioid Craving Scale prior to treatment and recording a score for said one or more surveys;
comparing said scores from one or more surveys to a reference value; and
administering a treatment regimen comprising a predetermined dose of a partial opioid agonist to treat the subject if (1) said score from one or more surveys is above the reference value; and (2) said one or more target catecholamine levels is greater than the reference value; or (3) said subject has elevated DNA methylation compared to the reference value used to determine if said subject has elevated DNA methylation.
(20)—A diagnostic kit for detecting autonomic dysfunction comprising, in a compartmentalized container, reagents, primers, oligonucleotides, and/or nucleic acid probes required for (1) performing a DNA methylation assay on human OPRM1 promoter region; and/or (2) performing an assay to determine catecholamine levels of one or more target catecholamines, on a biological sample.
(21)—A method for diagnosing if a subject has autonomic dysfunction comprising:
obtaining a biological sample from a subject;
assaying the biological sample to determine catecholamine levels of one or more target catecholamines;
comparing said one or more target catecholamine levels to a reference value; and
determining, based on the comparison of the target catecholamine level to the reference catecholamine value, that one or more target catecholamine levels is greater than the reference value;
wherein said subject has autonomic dysfunction when said target catecholamine level is greater than the reference value.
(22)—A method for diagnosing if a subject has autonomic dysfunction comprising:
a. obtaining a biological sample from a subject;
b. assaying the biological sample to determine catecholamine levels of one or more target catecholamines; and
c. comparing said one or more target catecholamine levels to a catecholamine reference value; and/or
d. conducting a DNA methylation assay on the biological sample to determine DNA methylation levels; and
e. comparing the results of said DNA methylation assay to a methylation reference value;
wherein said subject has autonomic dysfunction when said target catecholamine level is greater than the catecholamine reference value and/or when said DNA methylation levels are greater than the methylation reference value.

Many modifications and other embodiments of the invention set forth herein will come to mind to one skilled in the art to which this invention pertains having the benefit of the teachings presented in the descriptions herein. It will be appreciated by those skilled in the art that changes could be made to the embodiments described herein without departing from the broad inventive concept thereof. Therefore, it is understood that this invention is not limited to the particular embodiments disclosed, but it is intended to cover modifications within the spirit and scope of the present invention as defined by the included claims.

That which is claimed:

1. A method for treating a subject suffering from autonomic dysfunction comprising:
(a) obtaining a biological sample from a subject;
(b)(1) assaying the biological sample to determine catecholamine levels of one or more target catecholamines;
(b)(2) comparing said one or more target catecholamine levels to a reference catecholamine value;
(b)(3) determining, based on the comparison of the target catecholamine level to the reference catecholamine value, that one of more target catecholamine levels is greater than the reference catecholamine value; and/or
(c)(1) conducting a DNA methylation assay on the biological sample;
(c)(2) comparing the DNA methylation levels to a reference DNA methylation value;
(c)(3) determining, based on the comparison of DNA methylation levels to a reference DNA methylation value, that the subject has elevated DNA methylation levels; and
(d) administering a treatment regimen comprising a predetermined dose of a partial opioid agonist to treat the subject when said one or more target catecholamine levels is greater than the reference catecholamine value and/or when the subject has elevated DNA methylation.

2. The method of claim 1, wherein the one or more target catecholamines is selected from the group consisting of epinephrine and norepinephrine.

3. The method of claim 1, wherein said reference catecholamine value comprises a normal concentration range of said target catecholamine.

4. The method of claim 1, wherein the partial opioid agonist is buprenorphine.

5. The method of claim 4, wherein said predetermined dose of buprenorphine is 16 mg.

6. The method of claim 1, wherein the biological sample is blood.

7. The method of claim 1, wherein the biological sample is saliva.

8. The method of claim 1, wherein said reference DNA methylation value to determine if said subject has elevated DNA methylation comprises a normal control sample.

9. The method of claim 1, further comprising the step of:
(b)(4) analyzing the DNA methylation results using an unsupervised machine learning protocol to differentiate between normal control samples and samples with elevated DNA methylation levels.

10. The method of claim 9, wherein said unsupervised machine learning protocol is selected from Principal Component Analysis (PCA) and/or Non-Metric Dimensional Reduction Scaling (NMDS).

11. The method of claim 1, wherein the partial opioid agonist is buprenorphine.

12. The method of claim 11, wherein said predetermined dose of buprenorphine is 16 mg.

13. The method of claim 1, wherein the biological sample is blood.

14. The method of claim 1, wherein the biological sample is saliva.

15. The method of claim 1, wherein said DNA methylation assay is performed on the human OPRM1 promoter region.

16. The method of claim 15, wherein said DNA methylation assay is performed on the CpG Island region of the human OPRM1 promoter.

17. The method of claim 15, wherein said DNA methylation assay is performed on a plurality of specific CpG sites within the human OPRM1 promoter region.

18. The method of claim 1 wherein said subject has elevated DNA methylation levels when at least one of the CpG sites in FIG. 33 has elevated methylation compared to the reference DNA methylation value.

19. The method of claim 1, further comprising the steps of:
administering one or more surveys to the subject selected from Autonomic Dysfunction Scale and Opioid Craving Scale prior to treatment and recording a score for said one or more surveys;
comparing said scores from one or more surveys to a reference survey value;
determining based on the comparison of said scores to a reference survey value, that the subject has an elevated score; and
administering a treatment regimen comprising predetermined dose of a partial opioid agonist to treat the subject when (1) said one or more target catecholamine levels is greater than the reference catecholamine value; and/or (2) said subject has elevated DNA methylation levels compared to the reference DNA methylation value; and (3) said score from one or more surveys is above the reference survey value.

20. The method of claim 19 wherein said one or more surveys are selected from Autonomic Dysfunction Scale and/or Opioid Craving Scale.

21. The method of claim 20 wherein the Autonomic Dysfunction Scale comprises the following set of conditions that said test subject is asked to score: 1) I am yawning more than normal; 2) My eyes are watering more than normal; 3) My nose is running more than normal; 4) I am having stomach cramping; 5) I am vomiting; 6) I have diarrhea; 7) I am sweating more than normal; 8) The hair on my body is standing on end; 9) My heart is beating hard and fast; 10) I feel anxious; 11) I feel hot then cold; 12) I have a tremor (shaking); 13) 1 feel like something bad is about to happen; and 14) 1 can't stand feeling this way.

22. The method of claim 20 wherein the Opioid Craving Scale comprises the following set of conditions that the test subject is asked to score: 1) If I had an opioid right now, 1 would take it; 2) I would not be able to stop myself from taking an opioid right now; 3) I would feel more in control of things if I could take an opioid right now; 4) Taking an opioid right now would make me feel better; 5) If I could take an opioid right now I would feel less restless; 6) I am craving an opioid right now; and 7) Using an opioid right now would make me feel better.

23. A diagnostic kit for detecting autonomic dysfunction comprising, in a compartmentalized container, reagents, primers, oligonucleotides, and/or nucleic acid probes required for (1) performing a DNA methylation assay on human OPRM1 promoter region; and/or (2) performing an assay to determine catecholamine levels of one or more target catecholamines, on a biological sample.

24. A method for diagnosing if a subject has autonomic dysfunction comprising:
(a) obtaining a biological sample from a subject;
(b)(1) assaying the biological sample to determine catecholamine levels of one or more target catecholamines;
(b)(2) comparing said one or more target catecholamine levels to a catecholamine reference value; and
(b)(3) determining, based on the comparison of the target catecholamine level to the reference catecholamine value, that one of more target catecholamine levels is greater than the reference catecholamine value; and/or
(c)(1) conducting a DNA methylation assay on the biological sample to determine DNA methylation levels;
(c)(2) comparing the results of said DNA methylation assay to a reference DNA methylation value; and
(c)(3) determining, based on the comparison of DNA methylation levels to a reference DNA methylation value, that the subject has elevated DNA methylation levels;
d) wherein said subject has autonomic dysfunction when said target catecholamine level is greater than the reference catecholamine value and/or when said DNA methylation levels are greater than the reference DNA methylation value.
